SEQUENCE LISTING (11 sequences)

SEQ ID NO: 1 - DNA, 21 bp, Artificial Sequence
  Description of Artificial Sequence Synthetic probe
tgcgctcctggacgtagcctt

SEQ ID NO: 2 - DNA, 21 bp, Artificial Sequence
  Description of Artificial Sequence Synthetic probe
aaggctacgtccaggagcgca

SEQ ID NO: 3 - DNA, 21 bp, Artificial Sequence
  Description of Artificial Sequence Synthetic probe
aagcctacgtccaggagcgca

SEQ ID NO: 4 - DNA, 20 bp, Artificial Sequence
  Description of Artificial Sequence Synthetic probe
ttcgtacatctcgccctctt

SEQ ID NO: 5 - DNA, 21 bp, Artificial Sequence
  Description of Artificial Sequence Synthetic probe
tccagcgactcaatcttcctc

SEQ ID NO: 6 - DNA, 20 bp, Artificial Sequence
  Description of Artificial Sequence Synthetic primer
ccgcatcttcttttgcgtcg

SEQ ID NO: 7 - DNA, 21 bp, Artificial Sequence
  Description of Artificial Sequence Synthetic primer
gcccaatacgaccaaatccgt

SEQ ID NO: 8 - DNA, 21 bp, Artificial Sequence
  Description of Artificial Sequence Synthetic primer
ggaccctgtgagtagccagta

SEQ ID NO: 9 - DNA, 19 bp, Artificial Sequence
  Description of Artificial Sequence Synthetic primer
cagctccgacagatccagt

SEQ ID NO: 10 - DNA, 19 bp, Artificial Sequence
  Description of Artificial Sequence Synthetic primer
ctgcggctcgatcaactca

SEQ ID NO: 11 - DNA, 21 bp, Artificial Sequence
  Description of Artificial Sequence Synthetic primer
tccagcgactcaatcttcctc

(19) United States
(12) Patent Application Publication
(10) Pub. No.: US 2022/0136972 A1
(43) Pub. Date: May 5, 2022

(54) DUAL-ENHANCED RAMAN SCATTERING-BASED BIOMOLECULAR SENSING SYSTEM USING GRAPHENE-PLASMONIC HYBRID NANOARRAY AND METHODS OF USE THEREOF

(71) Applicant: Rutgers, The State University of New Jersey, New Brunswick, NJ (US)

(72) Inventors: Ki-Bum LEE, Monmouth Junction, NJ (US); Letao YANG, Edison, NJ (US)

(21) Appl. No.: 17/512,211

(22) Filed: Oct. 27, 2021

Related U.S. Application Data
(60) Provisional application No. 63/107,580, filed on Oct. 30, 2020.

(51) Int. Cl. G01N 21/65; G01N 33/543; C12Q 1/6876

(52) U.S. Cl. CPC G01N 21/658; C12Q 1/6876; G01N 33/54306

(57) ABSTRACT

A surface-enhanced Raman scattering (SERS) sensing system or platform and methods of using the same, where the platform comprises a graphene coated-homogeneous plasmonic metal hybrid array, which synergizes both electromagnetic mechanism (EM)- and chemical mechanism (CM)-based signal enhancement for achieving sensitive and reproducible detection of Raman signals. The system and methods of using such system or platform may be applied to the analyses of various bio/chemical molecules, such as but not limited to those found in cells, in a highly sensitive and selective manner.

### CROSS-REFERENCE TO RELATED APPLICATION

This application claims the benefit of and priority to U.S. Provisional Application No. 63/107,580, filed Oct. 30, 2020, which is incorporated herein by reference in its entirety.

### STATEMENT OF RIGHTS TO INVENTIONS MADE UNDER FEDERALLY SPONSORED RESEARCH

This invention was made with government support under Grant No. CHE-1429062 awarded by the National Science Foundation and Grant No. T32GM008339 awarded by the National Institutes of Health. The government has certain rights in the invention.

### SEQUENCE LISTING

The instant application contains a Sequence Listing which has been submitted electronically in ASCII format and is hereby incorporated by reference in its entirety. Said ASCII copy, created on Jan. 5, 2022, is named 117465-019002_US_SL.txt and is 2,875 bytes in size.

### FIELD OF DISCLOSURE

The present disclosure generally relates to methods and systems for detecting and analyzing molecules and biomolecular interactions.

### BACKGROUND

Biomolecular interactions within a cellular context play a vital role in various biological processes such as gene regulation and signal transduction, making them essential for disease diagnostics and drug screening. However, detecting and analyzing biomolecular interactions is typically difficult, as molecular interactions are governed by complicated thermodynamic processes and require structure-dependent recognition events to investigate them.

### SUMMARY

Embodiments of the present disclosure provide a system, comprising, consisting essentially of, or consisting of: a nanoarray, where the nanoarray may comprise, consist essentially of, or consist of: a substrate, wherein the substrate is glass; and a plurality of plasmonic metal protrusions extending from the substrate; where the plurality of plasmonic metal protrusions is of a plasmonic metal that is gold (Au); where the plasmonic metal in each of the plurality of plasmonic metal protrusions may have a thickness in a range of, from, including, and/or between 1 nm-200 nm (e.g., 20 nm-200 nm); where the plurality of plasmonic metal protrusions has a respective plurality of graphene oxide (GO) nanosheet coatings layered thereupon (i.e., the plurality of GO nanosheet coatings is layered on the plurality of plasmonic metal protrusions); where each of the GO nanosheet coatings has a lateral size in a range of, from, including, and/or between 10 nm-500 nm (e.g., 43 nm-295 nm). In one aspect of the embodiment, the sample comprises, consists essentially of, or consists of molecules or biochemical molecules applied to or located on the plurality of plasmonic metal protrusions. Another aspect of the embodiment may be directed to the biochemical molecules in the sample, where the biochemical molecules are selected from the group consisting of: cells, cell-derived vesicles, RNA sequences, DNA sequences, pathogens, antigens, viruses, and viral particles. A further aspect may provide a sample of molecules, where the molecules may be selected from, but not limited to, biochemical molecules; biomolecules; small molecules; drugs; nucleic acids (e.g., DNA, DNA strand, DNA sequence, cDNA, RNA, RNA strand, DNA sequence, mRNA, miRNA); cells (e.g., stem cells, neural stem cells, differentiating cells, cell-derived vesicles); proteins; metabolites, pathogen, antigen, virus or viral particle: influenza virus (e.g., influenza A, zoonotic influenza, influenza B), respiratory syncytial virus, parainfluenza virus, adenovirus, rhinovirus, metapneumovirus, human metapneumovirus and endemic human coronaviruses (e.g., HKU1, OC43, NL63, 229E), enterovirus (e.g., EVD68), and coronavirus (e.g., MERS-CoV, SARS-CoV, SARS-CoV-2 or 2019-nCoV). In yet another aspect, the system of embodiments of the disclosure may be directed to molecules in the sample, where the molecules or biochemical molecules comprise, consist essentially of, or consist of: i) a detectable label (e.g., a Raman dye (e.g., fluorophores (e.g., rhodamine X (ROX), rhodamine 6G, hexachlorofluorescein (HEX), 6-carboxyfluorescein (FAM), tetrachlorofluorescein (TET), sulfo-cyanine 3 (Cy3), sulfo-cyanine 5 (Cy5), tetramethyl rhodamine (TAMRA)) or the molecules or biochemical molecules are detectably labeled with any of the aforementioned exemplified labels; and ii) a coupling (e.g., covalent conjugation by, for example, a cysteamine linker) to at least one plasmonic metal protrusion or at least a portion of the plurality of plasmonic metal protrusions. Yet another aspect may be directed to biochemical molecules that emit at least one surface enhanced Raman scattering (SERS) light in response to a light directed onto the plurality of plasmonic metal protrusions from an incident light source.

One embodiment provides a system, comprising, consisting essentially of, or consisting of: a nanoarray, comprising, consisting essentially of, or consisting of: a substrate; a plurality of plasmonic metal protrusions extending from the substrate; where the plurality of plasmonic metal protrusions has a respective plurality of graphene oxide (GO) nanosheet coatings; a sample comprising biochemical molecules located on the plurality of plasmonic metal protrusions; where the biochemical molecules in the sample are: i) labeled with a Raman dye; and ii) coupled to at least a portion of the plurality of plasmonic metal protrusions; an incident light source configured to direct a light, having at least one excitation frequency, onto the plurality of plasmonic metal protrusions; where the biochemical molecules emit at least one surface enhanced Raman scattering (SERS) light in response to the light being directed onto the plurality of plasmonic metal protrusions from the incident light source; a detector configured to detect at least one laser power intensity and at least one Raman shift in vibrational wavenumber of the at least one Raman dye in a SERS signal spectrum or SERS spectra; where a signal-to-noise ratio (SNR) of the at least one SERS spectra is above a SNR predefined threshold when: a thickness of a plasmonic metal in each of the plurality of plasmonic metal protrusions is in a range between 20 nm to 200 nm; a lateral size of each of the GO nanosheet coatings is in a range between 43 nm and 295 nm; and a composition of the Raman dye is chosen to have a Raman cross-section value at the at least one excitation frequency greater than a 3×10¹⁴ Hz; and a processor configured to: i) receive, from the detector, data about the at least one Raman shift in vibrational wavenumber of the at least one Raman dye in a SERS spectra, the at least one laser power intensity of the at least one SERS spectra, or any combination thereof, and ii) identify the biochemical molecules in the sample based on the at least one Raman shift in vibrational wavenumber of the at least one Raman dye in a SERS spectra, the at least one laser power intensity of the at least one SERS spectra, or any combination thereof. In one aspect, the system of the embodiment provides the composition of the Raman dye, which comprises Cy5. Another aspect of the embodiment provides a sample, where the biochemical molecules in the sample are selected from the group consisting of: cells, cell-derived vesicles, RNA sequences, DNA sequences, pathogens, antigens, viruses, and viral particles. In a further aspect of the embodiment, the SNR predefined threshold is 34. Yet another aspect of the embodiment may be directed to each plasmonic metal protrusion extending from the substrate is cone-shaped. In some aspects of the embodiment, each plasmonic metal protrusion that is cone-shaped has a width of 250 nm and a height of 100 nm. A further aspect of the embodiment provides the substrate that is glass. In one aspect of the embodiment, the plasmonic metal is gold. Another aspect of the embodiment provides the plurality of GO nanosheet coatings of the plurality of plasmonic metal protrusions have a thickness in a range between 1 nm and 2 nm. In yet a further aspect of the embodiment, the biochemical molecules comprise neural stem cells, and where the processor is further configured to monitor changes in the SERS signal spectrum or SERS spectra for characterizing neural stem cell differentiation.

Another embodiment of the disclosure provides a method, comprising, consisting essentially of, or consisting of: disposing a sample onto a plurality of plasmonic metal protrusions extending from a substrate; where the plurality of plasmonic metal protrusions has a respective plurality of graphene oxide (GO) nanosheet coatings; where the sample comprises biochemical molecules that are coupled to at least a portion of the plurality of plasmonic metal protrusions; labeling the biochemical molecules in the sample with a Raman dye; illuminating the plurality of plasmonic metal protrusions with a light directed from an incident light source having at least one excitation frequency; where the biochemical molecules emit at least one surface enhanced Raman scattering (SERS) light in response to the light being directed onto the plurality of plasmonic metal protrusions from the incident light source; detecting, by a detector, at least one laser power intensity and at least one Raman shift in vibrational wavenumber of the at least one detectable label (e.g., Raman dye) or at least one detectable molecule (e.g., biomolecule, organic molecule analyte) in a SERS signal spectrum or SERS spectra; increasing a signal-to-noise ratio (SNR) of the at least one SERS spectra above a SNR predefined threshold by: varying a thickness of a plasmonic metal in the plurality of plasmonic metal protrusions to be within a range between 20 nm to 200 nm; varying a lateral size of each of the GO nanosheet coatings to be within a range between 43 nm and 295 nm; and choosing a composition of the Raman dye to have a Raman cross-section value at the at least one excitation frequency greater than 3×10¹⁴ Hz; receiving, by a processor, from the detector, data about the at least one Raman shift in vibrational wavenumber of the at least one Raman dye in a SERS spectra, the at least one laser power intensity of the at least one SERS spectra, or any combination thereof; and identifying, by the processor, the biochemical molecules in the sample based on the at least one Raman shift in vibrational wavenumber of the at least one Raman dye in a SERS spectra, the at least one laser power intensity of the at least one SERS spectra, or any combination thereof. In one aspect of the embodiment, the composition of the Raman dye comprises, consists essentially of, or consists of Cy5. Another aspect of the embodiment provides a sample comprising, consisting essentially of, or consisting of the biochemical molecules in the sample that are selected from the group consisting of: cells, cell-derived vesicles, RNA sequences, DNA sequences, pathogens, antigens, viruses, and viral particles. A further aspect of the embodiment may be directed to the substrate that is glass. In yet a further aspect of the embodiment, the plasmonic metal is gold. One aspect of the embodiment provides the method further comprises coating the plurality of plasmonic metal protrusions with GO nanosheets by applying electrostatic interactions using a chemical linker; where the plurality of plasmonic metal protrusions has a GO coating thickness in a range between 1 nm and 2 nm. In another aspect of the embodiment, the method further comprises forming the plurality of plasmonic metal protrusions on the substrate using laser interference lithography and a physical vapor deposition (PVD) of gold. One aspect of the embodiment provides the method, where forming the plurality of plasmonic metal protrusions using the laser interference lithography and the PVD of gold comprises forming each of the plurality of plasmonic metal protrusions that are cone-shaped. In a further aspect of the embodiment, where each cone-shaped plasmonic metal protrusion has a width of 250 nm and a height of 100 nm. In yet a further aspect of the embodiment, the biochemical molecules comprise neural stem cells, and further comprising monitoring, by the processor, changes in the SERS signal spectrum or SERS spectra for characterizing neural stem cell differentiation.

### BRIEF DESCRIPTION OF FIGURES

Various embodiments of the present disclosure can be further explained with reference to the attached drawings, wherein like structures are referred to by like numerals throughout the several views. The drawings shown are not necessarily to scale, with emphasis instead generally being placed upon illustrating the principles of the present disclosure. Therefore, specific structural and functional details disclosed herein are not to be interpreted as limiting, but merely as a representative basis for teaching one skilled in the art to variously employ one or more illustrative embodiments.

FIG. 1A shows an exemplary system of the disclosure, which is illustrative of some aspects of at least some embodiments of the present disclosure. FIG. 1B presents a field-emission scanning electron microscopy (FE-SEM) image of Au nanoarray that demonstrates a top view of a large-scale homogeneous plasmonic array composed of gold (Au)-coated PR nanocones. Scale bar: 1 μm. FIG. 1C illustrates a schematic flow diagram of an exemplary method of the disclosure, which is illustrative of some aspects of at least some embodiments of the present disclosure, of which the steps may occur simultaneously, sequentially, and/or separately.

FIGS. 2A-2D illustrate the development of homogeneous plasmonic Au nanoarrays to enhance Raman signal via the electromagnetic mechanism. FIG. 2A shows a schematic illustration of sequential steps to generate the Au nanoarray using laser interference lithography followed by gold deposition. FIG. 2B illustrates height profile of the Au nanoarray. FIG. 2C shows signal enhancement in relation to the thickness of Au (nm). FIG. 2D provides a Raman spectra of graphene-Au hybrid nanoarray at different excitation wavelength-dependent SERS effects, matching the trend predicted by FDTD simulation. Laser intensities at different wavelengths (514 nm, 633 nm, 780 nm) kept the same at 10% level.

FIGS. 3A-3G illustrate optimizing energy levels of graphene oxide to maximize Raman signal enhancement via a chemical mechanism on the plasmonic nanoarray. FIG. 3A shows a schematic diagram illustrating the combined electromagnetic and chemical mechanism obtained by graphene-Au hybrid nanoarray through tunable energy levels of graphene oxide (GO). FIG. 3B illustrates Raman spectra of synthesized GO and the graphene-Au hybrid nanoarray formed by electrostatic assembly. FIG. 3C illustrates a proposed mechanism for the reduction-dependent CM-based enhancement on the GO surface, which is mediated through the modulation of energy levels of GO: (i) charge transfer between rGO and Cy5, (ii) excitation of electrons from the hybridized rGO-Cy5 complex under the EM field, (iii) relaxation of excited electrons to the LUMO accompanied by Raman scattering, and (iv) electrons at the LUMO return to the ground state of rGO-cy5 complex. FIG. 3D presents Raman spectra of GO coated Au nanoarray after the reduction of GO under various reducing conditions [ascorbic acid (AA) and hydrazine] to form the graphene-Au hybrid nanoarray. FIG. 3E shows calculated carbon to oxygen ratios of rGO synthesized from various reducing conditions based on XPS spectra. FIG. 3F illustrates reduction time-dependent CM-based enhancement of Raman dye (Cy5) on the graphene-Au hybrid nanoarray. FIG. 3G provides dynamic light scattering (DLS) analysis of as-synthesized graphene oxide (GO). The average hydrodynamic size of GO was calculated to be 77.27 nm, which is suitable for forming the graphene-Au hybrid nanoarray.

FIGS. 4A-4G present applying graphene-Au hybrid nanoarray-based biosensors for monitoring stem cell neuronal differentiation. FIG. 4A shows a schematic diagram illustrating the binding and SERS-based detection of Raman dye (Cy5)-labeled DNA on the graphene-Au hybrid nanoarray. FIG. 4B illustrates a Raman spectrum of the graphene-Au hybrid nanoarray before (black line; ˜0 intensity) and after absorption of Cy5-labeled DNA (red; 0-12 intensity). FIG. 4C presents concentration-dependent changes of SERS signals at the characteristic peak of 1120 cm⁻¹ and their curve fitting on hybrid nanoarray absorbed with different amount of Cy5-labeled DNA. FIG. 4D shows a mechanism of graphene-Au hybrid nanoarray-based gene detection via the reversible adsorption and detachment of Raman dye-labeled probe DNA through complementary DNA hybridization. FIG. 4E illustrates representative Raman spectroscopy before and after the addition of the target complementary DNA sequence at different concentrations on the graphene-Au hybrid nanoarray. FIG. 4F presents a timeline of the neuronal differentiation from a human neural stem cell line. FIG. 4G illustrates RT-PCR experiments characterizes the neuronal differentiation of hNSCs after amplification of the DNA using PCR. FIG. 4H shows a graphene-Au hybrid nanoarray-based sensing of TuJ1 and GFAP obtained from hNSCs before (D1) and after neuronal differentiation (D15). FIG. 4I presents quantification of gene levels using graphene-Au hybrid nanoarray-based SERS sensing platform. Error bars represent standard deviation around the mean, n=3 from three independent measurements on three different substrates (FIGS. 4C, 4E, 4H, 4I) or from technical replicates (FIG. 4G). * and ** represent P<0.05 and 0.01, respectively by one-way Analysis of variance (ANOVA) and Tukey post hoc test. FIG. 4J provides a zeta potential of as-synthesized graphene oxide. The negatively charged graphene oxide can be readily coated to positively charged, amine-functionalized Au surfaces to form the hybrid nanoarray.

FIG. 5 shows a transmission electron microscopy (TEM) characterization of thin-layered graphene oxide (GO) on the gold nanoarray. The negatively charged graphene oxide with 1-3 layers thickness was functionalized to the cysteamine-conjugated gold nanoarray through electrostatic interactions.

FIG. 6A (left panel) illustrates X-ray photoelectron spectroscopy (XPS) of graphene oxide within the subsequent reduction. These XPS spectra indicate a time and reductant dependent reduction of graphene oxide (GO) (FIG. 6A; right panel) with 12 hours (FIG. 6B), 24 hours (FIG. 6C), and 48 hours (FIG. 6D) of ascorbic acid, and hydrazine (FIG. 6E), which was used for the modulation of graphene energy levels to achieve a maximum chemical enhancement of SERS. The summarized carbon to oxygen ratio from each condition of reduction can be found in FIG. 3G.

FIG. 7 presents additional Raman spectra illustrating the dual enhanced SERS mechanism from the graphene gold hybrid nanoarray. From the spectra, the most significant enhancement of Raman signals was found in the rGO/Au nanoarray of the disclosure, which is 16-fold and 31-fold higher than nanoarray alone or rGO alone, respectively, thereby validating the dual enhanced SErS.

FIGS. 8A-8D illustrate graphene-Au hybrid nanoarray-based SERS detection of methylene blue and turn-on detection of biomolecules (FIG. 8B) other than Cy5 and DNA via modulation of energy level and oxidation status of graphene oxide on the gold nanoarray. FIG. 8A presents graphene oxide controlled to be in its unreduced form to match the energy level of methylene blue (MB), a clinically used biomolecule for anti-oxidation and eye diseases. FIG. 8C shows repeated tests using large scale and homogeneous SERS. FIG. 8D illustrates a fingerprint analysis and reliable biomolecular detection.

FIGS. 9A-9B show a high signal-to-noise ratio and air stability of the SERS-based Cy5-DNA-labeled graphene-Au hybrid nanoarray detection platform. FIG. 9A illustrates time-dependent Raman spectra, which indicate the stable (red indicates the spectrum from sensor device initially fabricated and green indicates the spectrum from sensor incubated in the air) SERS signals after incubation in air. From these spectra, the high signal-to-noise ratio (SNR=34) of the sensing platform of the disclosure may be calculated and confirmed. FIG. 9B presents statistical analysis on the Raman intensity at Raman Shift used in the sensing application (1120 cm⁻¹) of the disclosure reveals no significant changes before and after air incubation.

FIG. 10 shows a deviation comparison between the repeated measurements on the same sample and measurements from the new sample preparation on each substrate. Three measurements on the same substrate were performed in the group of “Repeat measurement.” Three measurements from different substrates were performed in the group of “new sample.”

FIGS. 11A-11B illustrate selective detection of target DNA using the graphene-Au hybrid nanoarray. FIG. 11A presents a Raman mapping (peak position: 1120 cm⁻¹) of the Cy5-DNA probe-labelled graphene-Au hybrid nanoarray under different conditions (Control—red/black (Left), incubation with single-base pair mismatch target DNA (Mismatch—orange/yellow (Center), and positive for incubation with complementary DNA (Positive—purple (Right)). FIG. 11B shows randomly selected 50 spots at the hybrid nanoarray treated with three different conditions (i.e., Control, Mismatch, Positive) and their summarized Raman intensity at the Raman shift at 1120 cm⁻¹. These results suggest the ability of the SERS platform of the disclosure to selectively detect the target DNA at a single base pair mismatch level (red dots forming upper line: control; blue dots forming middle line: single base pair mismatch; and black dots forming lower line: positive control with complementary DNA sequence).

FIG. 12 presents NUPACK calculation on the melting point of the matched/mismatched pairing. Higher melting point of the matched pairing (black square, lower curve) to mismatched pairing (red circle, upper curve) calculated by NUPACK. (Zadeh et al. Journal of computational chemistry. 32:170-173, 2011).

FIG. 13 shows a table of the sequences for the molecular probe with Raman dye and genes (positive, single base pair mismatch noted by underlined text) analyzed for the cell-free condition. FIG. 13 discloses SEQ ID NOS 1-5, respectively, in order of appearance.

FIG. 14 illustrates the primer sequence for the genes (e.g., GAPDH, TuJ1, GFAP) analyzed by qRT-PCR. FIG. 14 discloses SEQ ID NOS 6-11, respectively, in order of appearance.

### DETAILED DESCRIPTION

Detailed embodiments of the present disclosure, taken in conjunction with the accompanying figures, are disclosed herein; however, it is to be understood that the disclosed embodiments are merely illustrative of the disclosure that may be embodied in various forms. In addition, each of the examples given in connection with the various embodiments of the disclosure is intended to be illustrative, and not restrictive.

All terms used herein are intended to have their ordinary meaning in the art unless otherwise provided. All concentrations are in terms of percentage by weight of the specified component relative to the entire weight of the topical composition, unless otherwise defined.

The phrases “in one embodiment” and “in some embodiments” as used herein do not necessarily refer to the same embodiment(s), though it may. Furthermore, the phrases “in another embodiment” and “in some other embodiments” as used herein do not necessarily refer to a different embodiment, although it may. Thus, as described below, various embodiments may be readily combined, without departing from the scope or spirit of the present disclosure.

In addition, the term “based on” is not exclusive and allows for being based on additional factors not described, unless the context clearly dictates otherwise. As used herein, “a,” “an,” or “the” shall mean one or more, i.e., include plural references. As used herein when used in conjunction with the word “comprising,” the words “a” or “an” mean one or more than one. The meaning of “in” includes “in” and “on.” As used herein “another” means at least a second or more.

As used herein, all ranges of numeric values include the endpoints and all possible values disclosed between the disclosed values. The exact values of all half-integral numeric values are also contemplated as specifically disclosed and as limits for all subsets of the disclosed range. For example, a range of from 0.1% to 3% specifically discloses a percentage of 0.1%, 1%, 1.5%, 2.0%, 2.5%, and 3%. Additionally, a range of 0.1 to 3% includes subsets of the original range including from 0.5% to 2.5%, from 1% to 3%, from 0.1% to 2.5%, etc. It will be understood that the sum of all weight % of individual components will not exceed 100%.

Throughout this description, various components may be identified having specific values or parameters, however, these items are provided as exemplary embodiments. Indeed, the exemplary embodiments do not limit the various aspects and concepts of the present disclosure as many comparable parameters, sizes, ranges, and/or values may be implemented. Unless otherwise specified, the terms “first,” “second,” and the like, “primary,” “secondary,” and the like, do not denote any order, quantity, or importance, but rather are used to distinguish one element from another.

By “consist essentially” it is meant that the ingredients include only the listed components along with the normal impurities present in commercial materials and with any other additives present at levels which do not affect the operation of the disclosure, for instance at levels less than 5% by weight or less than 1% or even 0.5% by weight.

Typically, alkyl groups described herein refer to a branched or straight-chain monovalent saturated aliphatic hydrocarbon radical of 1-30 carbon atoms (e.g., 1-16 carbon atoms, 6-20 carbon atoms, 8-16 carbon atoms, or 4-18 carbon atoms, 4-12 carbon atoms, etc.). In some embodiments, the alkyl group may be substituted with 1, 2, 3, or 4 substituent groups as defined herein. Alkyl groups may have 1-26 carbon atoms. In other embodiments, alkyl groups will have from 6-18 or from 1-8 or from 1-6 or from 1-4 or from 1-3 carbon atoms, including for example, embodiments having one, two, three, four, five, six, seven, eight, nine, or ten carbon atoms. Any alkyl group may be substituted or unsubstituted. Examples of alkyl groups include methyl, ethyl, propyl, butyl, pentyl, hexyl, heptyl, octyl, nonyl, decyl, undecyl, and dodecyl groups. Heteroalkyl groups may refer to branched or straight-chain monovalent saturated aliphatic hydrocarbon radicals with one or more heteroatoms (e.g., N, O, S, etc.) in the carbon chain. Heteroalkyl groups may have 1-30 carbon atoms (e.g., 1-16 carbon atoms, 6-20 carbon atoms, 8-16 carbon atoms, or 4-18 carbon atoms, 4-12 carbon atoms, etc.). In some embodiments, the heteroalkyl group may be substituted with 1, 2, 3, or 4 substituent groups as defined herein. Heteroalkyl groups may have from 1-26 carbon atoms. In other embodiments, heteroalkyl groups will have from 6-18 or from 1-8 or from 1-6 or from 1-4 or from 1-3 carbon atoms, including for example, embodiments having one, two, three, four, five, six, seven, eight, nine, or ten carbon atoms. In some embodiments, the heteroalkyl group can be further substituted with 1, 2, 3, or 4 substituent groups as described herein for alkyl groups. Examples of heteroalkyl groups are an alkoxy. Alkoxy substituent groups or alkoxy-containing substituent groups may be substituted by, for example, one or more alkyl groups.

Aryl groups may be aromatic mono- or polycyclic radicals of 6 to 12 carbon atoms having at least one aromatic ring. Examples of such groups include, but are not limited to, phenyl, naphthyl, 1,2,3,4-tetrahydronaphthalyl, 1,2-dihydronaphthalyl, indanyl, and 1H-indenyl. Typically, heteroaryls include mono- or polycyclic radical of 5 to 12 atoms having at least one aromatic ring containing one, two, or three ring heteroatoms selected from N, O, and S, with the remaining ring atoms being C. One or two ring carbon atoms of the heteroaryl group may be replaced with a carbonyl group. Examples of heteroaryl groups are pyridyl, benzooxazolyl, benzoimidazolyl, and benzothiazolyl.

The term “substituent” refers to a group “substituted” on, e.g., an alkyl, at any atom of that group, replacing one or more hydrogen atoms therein (e.g., the point of substitution). In some aspects, the substituent(s) on a group are independently any one single, or any combination of two or more of the permissible atoms or groups of atoms delineated for that substituent. In another aspect, a substituent may itself be substituted with any one of the substituents described herein. Substituents may be located pendant to the hydrocarbon chain.

A substituted hydrocarbon group may have as a substituent one or more hydrocarbon radicals, substituted hydrocarbon radicals, or may comprise one or more heteroatoms. Examples of substituted hydrocarbon radicals include, without limitation, heterocycles, such as heteroaryls. Unless otherwise specified, a hydrocarbon substituted with one or more heteroatoms will comprise from 1-20 heteroatoms. In other embodiments, a hydrocarbon substituted with one or more heteroatoms will comprise from 1-12 or from 1-8 or from 1-6 or from 1-4 or from 1-3 or from 1-2 heteroatoms. Examples of heteroatoms include, but are not limited to, oxygen, nitrogen, sulfur, phosphorous, halogen (e.g., F, Cl, Br, I, etc.), boron, silicon, etc. In some embodiments, heteroatoms will be selected from the group consisting of oxygen, nitrogen, sulfur, phosphorous, and halogen (e.g., F, Cl, Br, I, etc.). In some embodiments, a heteroatom or group may substitute a carbon. In some embodiments, a heteroatom or group may substitute a hydrogen. In some embodiments, a substituted hydrocarbon may comprise one or more heteroatoms in the backbone or chain of the molecule (e.g., interposed between two carbon atoms, as in “oxa”). In some embodiments, a substituted hydrocarbon may comprise one or more heteroatoms pendant from the backbone or chain of the molecule (e.g., covalently bound to a carbon atom in the chain or backbone, as in “oxo”).

In addition, the phrase “substituted with a[n],” as used herein, means the specified group may be substituted with one or more of any or all of the named substituents. For example, where a group, such as an alkyl or heteroaryl group, is “substituted with an unsubstituted C₁-C₂₀ alkyl, or unsubstituted 2 to 20 membered heteroalkyl,” the group may contain one or more unsubstituted C₁-C₂₀ alkyls, and/or one or more unsubstituted 2 to 20 membered heteroalkyls. Moreover, where a moiety is substituted with an R substituent, the group may be referred to as “R-substituted.” Where a moiety is R-substituted, the moiety is substituted with at least one R substituent and each R substituent is optionally different.

Unless otherwise noted, all groups described herein (e.g., alkyl, cycloalkyl, heteroalkyl, heterocycloalkyl, aryl, heteroaryl, alkylene, heteroalkylene, cylcoalkylene, heterocycloalkylene, etc.) may optionally contain one or more common substituents, to the extent permitted by valency. Common substituents include halogen (e.g., F, Cl, etc.), C₁₋₁₂ straight chain or branched chain alkyl, C₂₋₁₂ alkenyl, C₂₋₁₂ alkynyl, C₃₋₁₂ cycloalkyl, C₆₋₁₂ aryl, C₃₋₁₂ heteroaryl, C₃₋₁₂ heterocyclyl, C₁₋₁₂ alkylsulfonyl, nitro, cyano, —COOR, —C(O)NRR′, —OR, —SR, —NRR′, and oxo, such as mono- or di- or tri-substitutions with moieties such as halogen, fluoroalkyl, perfluoroalkyl, perfluroalkoxy, trifluoromethoxy, chlorine, bromine, fluorine, methyl, methoxy, pyridyl, furyl, triazyl, piperazinyl, pyrazoyl, imidazoyl, and the like, each optionally containing one or more heteroatoms such as halo, N, O, S, and P. R and R′ are independently hydrogen, C₁₋₁₂ alkyl, C₁₋₁₂ haloalkyl, C₂₋₁₂ alkenyl, C₂₋₁₂ alkynyl, C₃₋₁₂ cycloalkyl, C₄₋₂₄ cycloalkylalkyl, C₆₋₁₂ aryl, C₇₋₂₄ aralkyl, C₃₋₁₂ heterocyclyl, C₃₋₂₄ heterocyclylalkyl, C₃₋₁₂ heteroaryl, or C₄₋₂₄ heteroarylalkyl. Further, as used herein, the phrase optionally substituted indicates the designated hydrocarbon group may be unsubstituted (e.g., substituted with H) or substituted. Typically, substituted hydrocarbons are hydrocarbons with a hydrogen atom removed and replaced by a substituent (e.g., a common substituent).

It is understood by one of ordinary skill in the chemistry art that substitution at a given atom is limited by valency. The use of a substituent (radical) prefix names such as alkyl without the modifier optionally substituted or substituted is understood to mean that the particular substituent is unsubstituted. However, the use of haloalkyl without the modifier optionally substituted or substituted is still understood to mean an alkyl group, in which at least one hydrogen atom is replaced by halo. Where a group may be substituted by one or more of a number of substituents, such substitutions are selected so as to comply with principles of chemical bonding with regard to valencies, etc., and to give compounds which are not inherently unstable. For example, any carbon atom will be bonded to two, three, or four other atoms, consistent with the four valence electrons of carbon. Additionally, when a structure has less than the required number of functional groups indicated, those carbon atoms without an indicated functional group are bonded to the requisite number of hydrogen atoms to satisfy the valency of that carbon.

As used herein, the term “subject” refers to any organism to which a composition and/or compound in accordance with the disclosure may be administered, e.g., for experimental, diagnostic, prophylactic, and/or therapeutic purposes. Typical subjects include any animal (e.g., mammals such as mice, rats, rabbits, non-human primates, dogs, cats, horses, and humans, etc.). A subject in need thereof is typically a subject for whom it is desirable to treat a disease, disorder, or condition as described herein. For example, a subject in need thereof may seek or be in need of treatment, require treatment, be receiving treatment, may be receiving treatment in the future, or a human or animal that is under care by a trained professional for a particular disease, disorder, or condition.

The terms “polynucleotide” or “nucleic acid,” as used herein, generally refer to molecules comprising a plurality of nucleotides. Exemplary polynucleotides include deoxyribonucleic acids, ribonucleic acids, and synthetic analogues thereof, including peptide nucleic acids. The term “nucleic acid” or “nucleic acid molecule,” as used herein, can include, for example, genomic DNA, cDNA, ssDNA, dsDNA, RNA, mRNA, tRNA, shRNA, rRNA, snRNA, miRNA, tmRNA, dsRNA and DNA-RNA hybrid molecules.

A “protein,” “peptide,” and “polypeptide” are used interchangeably herein and refer to a polymer of amino acid residues linked together by peptide (amide) bonds.

By “fragment” is meant a portion of a polypeptide or nucleic acid molecule. This portion contains at least 10%, 20%, 30%, 40%, 50%, 60%, 70%, 80%, or 90% of the entire length of the reference nucleic acid molecule or polypeptide. A fragment may contain any portion of a polypeptide or nucleic acid molecule or sequence, such as but not limited to, 3, 4, 5, 10, 20, 30, 40, 50, 60, 70, 80, 90, or 100, 200, 300, 400, 500, 600, 700, 800, 900, or 1000 nucleotides or amino acids.

“Hybridization” means hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds. By “hybridize” is meant pairing to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than 750 mM NaCl and 75 mM trisodium citrate, less than 500 mM NaCl and 50 mM trisodium citrate, and less than 250 mM NaCl and 25 mM trisodium citrate. Low stringency hybridization can be obtained in the absence of organic solvent, e.g., formamide, while high stringency hybridization can be obtained in the presence of at least 35% formamide, and at least 50% formamide. Stringent temperature conditions will ordinarily include temperatures of at least 30° C., of at least 37° C., and of at least 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In one embodiment, hybridization will occur at 30° C. in 750 mM NaCl, 75 mM trisodium citrate, and 1% SDS. In one embodiment, hybridization will occur at 37° C. in 500 mM NaCl, 50 mM trisodium citrate, 1% SDS, 35% formamide, and 100 μg/ml denatured salmon sperm DNA (ssDNA). In another embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will be less than 30 mM NaCl and 3 mM trisodium citrate, and less than 15 mM NaCl and 1.5 mM trisodium citrate. Stringent temperature conditions for the wash steps will ordinarily include a temperature of at least 15° C., of at least 42° C., and of at least 68° C. In one embodiment, wash steps will occur at 15° C. in 30 mM NaCl, 3 mM trisodium citrate, and 0.1% SDS. In another embodiment, wash steps will occur at 42 C in 15 mM NaCl, 1.5 mM trisodium citrate, and 0.1% SDS. In yet a further embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “fragment” is meant a portion of a polypeptide or nucleic acid molecule. This portion contains at least 10%, 20%, 30%, 40%, 50%, 60%, 70%, 80%, or 90% of the entire length of the reference nucleic acid molecule or polypeptide. A fragment may contain any portion of a polypeptide or nucleic acid molecule or sequence, such as but not limited to, 3, 4, 5, 10, 20, 30, 40, 50, 60, 70, 80, 90, or 100, 200, 300, 400, 500, 600, 700, 800, 900, or 1000 nucleotides or amino acids.

By “inhibitory nucleic acid” is meant a double-stranded RNA, siRNA, shRNA, or antisense RNA, or a portion thereof, or a mimetic thereof, that when administered to a mammalian cell results in a decrease (e.g., by 10%, 25%, 50%, 75%, or even 90-100%) in the expression of a target gene. Typically, a nucleic acid inhibitor comprises at least a portion of a target nucleic acid molecule, or an ortholog thereof, or comprises at least a portion of the complementary strand of a target nucleic acid molecule. For example, an inhibitory nucleic acid molecule comprises at least a portion of any or all of the nucleic acids delineated herein.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Such a sequence is at least 60% (e.g., 75%, 80%, 85%, 90%, 95%, 97%, 99%) identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

Raman spectroscopy is a technique for detecting specific molecules of interest, as well as biomolecular or chemical molecular interactions within cells, which has high specificity, sensitivity, and selectivity for chemical structures. This technique generally determines vibrational modes of molecules although rotational and other low-frequency modalities may also be used. Chemical molecules may be identified by a structural fingerprint using Raman spectroscopy as molecules have individual vibrational patterns. There are several uses for Raman spectroscopy, including but not limited to, in Pharmaceuticals (e.g., drug discovery, counterfeit pharmaceutical identification, contaminant identification, microfluidic device development); Materials Science (e.g., carbon nanotubes, diamond films); forensics (e.g., explosives, drugs of abuse, polymers/plastics, paint chips, ink); Semiconductor (e.g., contaminant identification, silicon strain, silicon crystal state, thin film quality control, materials research, OLED development, diamond-like films); Geology (e.g., gemstone identification, petroleum analysis, geological research); and Art (e.g., pigment identification on paintings, dye identification on textiles, development of pigments, development of dyes). Raman spectrum of molecules may have a specific configuration as represented by Raman bands. For example, the Raman spectrum of a crystal having a very defined lattice structure of identical atoms has a single dominant Raman band since there is one molecular environment of the crystal. Although Raman bands are highly specific, their signals are inherently weak, thus limiting the wide use of Raman spectroscopy in biosensing applications. The present disclosure describes the challenges to ensure high sensitivity and selectivity, while achieving uniform signal enhancement and high reproducibility for quantitative detection of targeted biomarkers within complex cell microenvironments, such as but not limited to, stem cells, neural cells, and the like.

It is recognized that Raman signals can be amplified using roughened metal (e.g., gold (Au), silver (Ag), platinum (Pt), aluminum (Al), copper (Cu), palladium (Pd), tin (Sn), germanium (Ge), bismuth (Bi), sodium (Na), lithium (Li), magnesium (Mg)) nanostructures as underlying substrates. The substrate may be any material (e.g., glass, silicon, plastics, fabrics/textiles, smart phone displays (e.g., aluminosilicate glass, alkali-aluminosilicate sheet glass, indium tin oxide)) that allows for surface roughening. This practice is known as surface-enhanced Raman scattering (SERS). One embodiment may provide for substrates with at least one structure or a plurality of structures (e.g., nanostructures, microstructures) that protrudes from the substrate surface, forming a large-scale, homogeneous array (e.g., polymer, nanoarray, microarray) deposited with at least one layer of plasmonic metal (e.g., Au, Ag, Pt, Al, Cu, Pd) in a thickness ranging from 1 nm-200 nm (e.g., 2 nm-200 nm, 3 nm-100 nm, 4 nm-80 nm, 5 nm-70 nm, 6 nm-60 nm, 7 nm-50 nm, 8 nm-40 nm, 9 nm-30 nm, 10 nm-20 nm) that is further uniformly layered with a 2D material (e.g., nanomaterial, graphene oxide (e.g., less than 300 nm)), where the lateral size may be 30 nm or greater (e.g., 35 nm, 40 nm, 45 nm, 50 nm, 75 nm, 100 nm, 125 nm, 150 nm, 200 nm, 225 nm, 250 nm, 275 nm, 295 nm), 300 nm or less (e.g., 295 nm, 275 nm, 250 nm, 200 nm, 175 nm, 150 nm, 125 nm, 100 nm, 75 nm, 50 nm, 25 nm), or in a range of 30 nm-300 nm (e.g., 40 nm-295 nm, 45 nm-275 nm, 50 nm-250 nm, 60 nm-200 nm, 75 nm-175 nm, 100 nm-150 nm).

The enhancement of Raman signals on SERS platforms can be achieved by different mechanisms, for example, an electromagnetic mechanism (EM) or a chemical mechanism (CM) both of which play a role for advanced biosensing applications. Conventional techniques for SERS-based biosensing platforms lack uniform distribution of noble metal nanostructures in the large-scale, which can lead to irreproducible EM-based enhancement. Thereby, such techniques are not suitable for comprehensive and quantitative analysis of biomolecules in cell biology. Similarly, mismatched surface energy levels of the nanostructures and adsorbates (i.e., molecules of interest) can significantly reduce the CM-based enhancement by limiting charge transfer, thus hindering the practical applications of SERS-based sensing. Addressing those critical issues, 2D nanomaterials, such as graphene, have emerged as plasmon-free substrates in SERS-based biosensing by improving CM-based enhancement through enabling charge transfer and stronger binding toward analytes. Although CM-based enhancement is highly dependent on the electronic structures of both the analytes and substrates, the performance of 2D nanomaterial-enhanced SERS can vary significantly for different molecules. In this regard, tuning the oxidation and the composition of graphene-like 2D nanomaterials can be an effective strategy for modulating energy levels, thus facilitating CM-based enhancement by better matching the energy levels of the analytes.

The embodiments described herein are directed to a SERS-based sensing system that provides highly homogeneous EM-based enhancement as well as a finely tuned CM-based enhancement for sensitive and quantitative biosensing applications. Such a SERS-based sensing system can orthogonally modulate the EM and the CM to achieve a dual-enhanced Raman scattering toward specific analytes.

In one embodiment of the disclosure, an ideal SERS-based sensing system should have both a highly homogenous EM-based enhancement as well as a finely tuned CM-based enhancement for sensitive and quantitative biosensing applications. Some embodiments of the disclosure provide for the design and development of a platform that orthogonally modulates both the EM and CM to result in a dual-enhanced Raman scattering toward specific analytes. An embodiment of the disclosure provides a novel sensing platform using graphene-coated homogeneous plasmonic metal (e.g., Au, Ag, Pt, Al, Cu, Pd) arrays (e.g., nanoarrays, microarrays) that may be used in the detection system and methods described here. The platform and specific arrays synergize both electromagnetic mechanism- and chemical mechanism-based enhancement.

Some embodiments may provide a surface-enhanced Raman scattering (SERS) sensing platform using graphene coated-homogenous plasmonic metal (e.g., Au) nanoarray, which synergize both electromagnetic mechanism (EM)- and chemical mechanism (CM)-based enhancement for sensitive and reproducible detection of Raman signals. Amplified Raman signals may be obtained by an optimized CM through the graphene-functionalized surface which aligns the energy level of the graphene oxide with the target molecule by tuning its oxidation levels. The unique surface properties of reduced graphene oxide (rGO) may simultaneously allow the target-specific detection of, for example, cell-derived biomolecules such as DNA and RNA in complex biological fluids (e.g., blood, saliva, urine, semen). The dissociation of DNA probes labeled with a label (e.g., Raman dye) upon hybridization of target DNA/RNA leads to a reduction of the Raman signal with a sensitivity in a range of, from, including, and/or between aM (10⁻¹⁸) to μM (10⁻⁶).

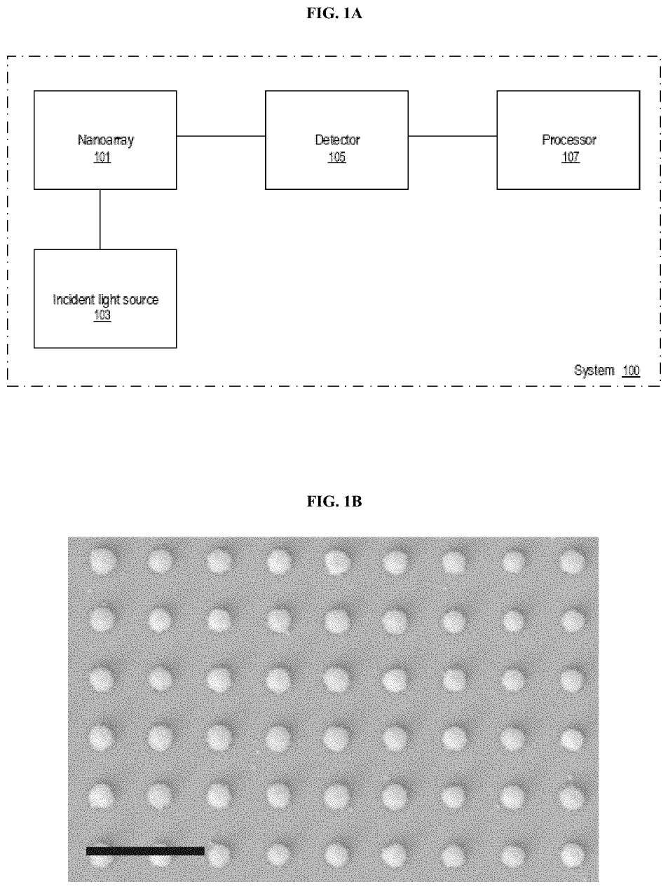

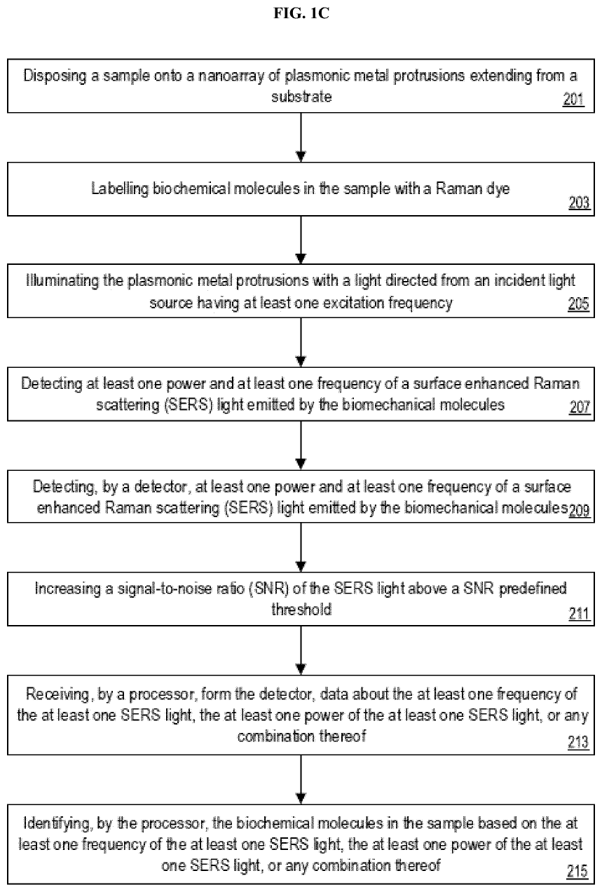

Another embodiment may be directed to a SERS-based sensing platform comprising a large-scale, homogeneous graphene-metal hybrid array encompassing both EM- and CM-based enhancement synergistically. An exemplary system (100) results in a combined Raman signal enhancement may be obtained by both electromagnetic and chemical mechanisms illustrated in FIG. 1A. A nanoarray (101) may be used to detect a molecule of interest in a sample when disposed thereon. The molecule of interest may be labeled with a Raman dye and detected in the detector (105) when the incident light source (103) applied to the nanoarray (101) illuminates the plasmonic metal protrusions resulting in at least one laser power intensity and at least one excitation frequency of a surface-enhanced Raman scattering (SERS) light emitted by the molecules of interest performed in a processor (107). An exemplary graphene-Au hybrid SERS nanoarray results from a large-scale, homogeneous nanoarray with a weak Raman signal; electromagnetic mechanism-based signal enhancement resulting from a plasmonic Au nanoarray with a strong Raman signal; and chemical mechanism-based signal enhancement resulting from dual-enhanced Raman from EM/CM using a graphene-Au hybrid nanoarray having the strongest Raman signal. The hybrid nanoarray allowing for a boosted Raman signal occurs via graphene oxide which enables tunable energy level. For the characterization of neuronal differentiation, sensitive and selective SERS-based gene detection may occur through a graphene-Au hybrid nanoarray which results in efficient adhesion via π-π staking interaction, specific labelling for gene detection on a SERS-active graphene-Au hybrid nanoarray detects the neuronal differentiation of neural stem cells (NSCs) to neurons by selective detection of stem cell derived genes which results in selective release via DNA hybridization.

The flowchart of FIG. 1B illustrates the steps for identifying biochemical molecules in a sample, where a sample is disposed onto a nanoarray of plasmonic metal protrusions extending from a substrate (201), Raman dye is used to label biochemical molecules in the sample (203), the plasmonic metal protrusions are illuminated with a light directed from an incident light source having at least one excitation frequency (205), detection, by a detector, of at least one laser power intensity and at least one Raman shift in vibrational wavenumber of a surface-enhanced Raman scattering (SERS) light emitted by the biochemical molecules (207), increasing a signal-to-noise ratio (SNR) of the SERS light above a SNR pre-defined threshold (SNR=34) (211), receiving, by a processor, from the detector, data about the at least one Raman shift in vibrational wavenumber of the at least one SERS spectra, the at least one laser power intensity of the at least one SERS spectra, or any combination thereof (213), and identifying, by the processor, the biochemical molecules in the sample based on the at least one Raman shift in vibrational wavenumber of the at least one Raman dye in a SERS spectra, the at least one laser power intensity of the at least one SERS spectra, or any combination thereof (215).

Signal-to-noise ratio (SNR) may be used as a parameter for sensing applications, such as the system of the disclosure that may be applied or used as a biosensor or in a biosensing application. However, absolute signal intensity may be a more direct metric for electromagnetic mechanism and chemical mechanism enhancement. In one embodiment of the disclosure, a factor for determining chemical mechanism enhancement may include, but is not limited to, the reduction rate of graphene oxide. The rate of reduction may be reversibly proportional to the amount of oxygen groups on the GO backbone, which may be defined as the carbon to oxygen ratio at 1 or greater (e.g., 1.1, 1.2, 1.3, 1.4, 1.5, 1.6, 1.7, 1.8, 1.9, 2, 2.1, 2.2, 2.3, 2.4, 2.5, 3, 3.5, 4, 4.5, 5); at 10 or less (e.g., 9.5, 9, 8.5, 8, 7.5, 7, 6.5, 6, 5.5, 5, 4.5, 4, 3.5, 3, 2.5, 2, 1.5, 1); or in a range of, from, including, and/or between 1-10 (e.g., 2-9, 3-8, 4-7, 5-6, 1.2-4.49).

### System

One embodiment of the disclosure provides a system, comprising, consisting essentially of, or consisting of: an array, which may include but is not limited to, a nanoarray or microarray. This array may comprise, consist essentially of, or consist of: a substrate; and at least one or a plurality of plasmonic metal (e.g., Au, Ag, Pt, Al, Cu, Pd, Ge, Bi, Mg, Na, Li, Sn) protrusions (e.g., nanostructures) extending from the substrate or the surface of the substrate, where the protrusions may have essentially any shape that promotes enhanced Raman shift, such as, but not limited to, cones, obelisks, pyramids, inverted funnels, wedges, cylinders, rings, trees, branches, rods, wires, sheets, flowers, urchins, triangles, diamonds, stars, or the like. Some aspects of the embodiment may provide for the at least one or the plurality of plasmonic metal protrusions having at least one or a plurality of respective two-dimensional (2D) material (e.g., nanomaterial, graphene oxide (GO), graphene, graphane, graphyne, 2D boron nitride, borophene, germanene, silicene, Si₂BN, metal chalcogens, manganese dioxide, perovskites, black phosphor, stanene, plumbene, phosphorene, antimonene, bismuthene, platinum, palladium, rhodium, 2D alloys, 2D crystals) sheet (e.g., nanosheet) coatings or layers of at least one or a plurality of layers (e.g., 2 layers, 3 layers, 4 layers, 5 layers, 10 layers, 20 layers).

In some embodiments, the system or platform of the disclosure may be used to identify or detect at least one molecule (e.g., biochemical molecule; biomolecule; small molecule; drug; nucleic acids (e.g., DNA, DNA strand, DNA sequence, cDNA, RNA, RNA strand, DNA sequence, mRNA, miRNA); cell (e.g., stem cell, neural stem cell, differentiating cells, cell-derived vesicles); proteins; metabolites, pathogen, antigen, virus or viral particle: influenza virus (e.g., influenza A, zoonotic influenza, influenza B), respiratory syncytial virus, parainfluenza virus, adenovirus, rhinovirus, metapneumovirus, human metapneumovirus and endemic human coronaviruses (e.g., HKU1, OC43, NL63, 229E), enterovirus (e.g., EVD68), and coronavirus (e.g., MERS-CoV, SARS-CoV, SARS-CoV-2 or 2019-nCoV)) or a plurality of molecules. The molecules of interest may be found in a sample, where the sample may be, for example, a complex cell microenvironment. In some embodiments, the sample may be selected from: blood, saliva, urine, semen, or samples comprising, consisting essentially of, or consisting of at least one molecule isolated or extracted from: hair, tissue, bone, teeth, and the like, of a subject (e.g., mammal) or soil, textile, glass, and the like.

One embodiment provides a system, comprising, consisting essentially of, or consisting of: an array (e.g., nanoarray, microarray), where the array may comprise, consist essentially of, or consist of: a substrate, wherein the substrate is selected from, but not limited to, glass, silicon, plastics, fabrics/textiles, smart phone displays (e.g., aluminosilicate glass, alkali-aluminosilicate sheet glass, indium tin oxide); and a plurality of plasmonic metal protrusions extending from the substrate; where the plurality of plasmonic metal protrusions is of a plasmonic metal (e.g., gold (Au), silver (Ag), platinum (Pt), aluminum (Al), copper (Cu), palladium (Pd), tin (Sn), germanium (Ge), bismuth (Bi), sodium (Na), lithium (Li), magnesium (Mg)); where the plasmonic metal in each of the plurality of plasmonic metal protrusions may have a thickness in a range of, from, including, and/or between 10 nm-20,000 nm (e.g., 20 nm-15,500 nm, 50 nm-15,000 nm, 100 nm-12,500 nm, 500 nm-10,500 nm); where the plurality of plasmonic metal protrusions has a respective plurality of graphene oxide (GO) nanosheet coatings layered thereupon (i.e., the plurality of GO nanosheet coatings is layered on the plurality of plasmonic metal protrusions); where each of the GO nanosheet coatings has a lateral size in a range of, from, including, and/or between 1 nm-20,000 nm (e.g., e.g., 1 nm-500 nm, 5 nm-20,000 nm, 7 nm-497 nm, 9 nm-397 nm, 10 nm-500 nm, 13 nm-297 nm, 23 nm-295 nm, 33 nm-197 nm, 43 nm-195 nm, 53 nm-97 nm, 63 nm-95 nm, 73 nm-87 nm; 43 nm-295 nm). In one aspect of the embodiment, the sample comprises, consists essentially of, or consists of molecules or biochemical molecules applied to or located on the plurality of plasmonic metal protrusions. Another aspect of the embodiment may be directed to the biochemical molecules in the sample, where the biochemical molecules are selected from the group consisting of: cells, cell-derived vesicles, RNA sequences, DNA sequences, pathogens, antigens, viruses, and viral particles. A further aspect may provide a sample of molecules, where the molecules may be selected from, but not limited to, biochemical molecules; biomolecules; small molecules; drugs; nucleic acids (e.g., DNA, DNA strand, DNA sequence, cDNA, RNA, RNA strand, DNA sequence, mRNA, miRNA); cells (e.g., stem cells, neural stem cells, differentiating cells, cell-derived vesicles); proteins; metabolites, pathogen, antigen, virus or viral particle: influenza virus (e.g., influenza A, zoonotic influenza, influenza B), respiratory syncytial virus, parainfluenza virus, adenovirus, rhinovirus, metapneumovirus, human metapneumovirus and endemic human coronaviruses (e.g., HKU1, OC43, NL63, 229E), enterovirus (e.g., EVD68), and coronavirus (e.g., MERS-CoV, SARS-CoV, SARS-CoV-2 or 2019-nCoV). In yet one aspect, the system of embodiments of the disclosure may be directed to molecules in the sample, where the molecules or biochemical molecules comprise, consist essentially of, or consist of: i) a detectable label (e.g., marker, biomarker, dye, or the like, or composition thereof, such as but not limited to a Raman dye (e.g., fluorophores (e.g., rhodamine X (ROX), rhodamine 6G, hexachlorofluorescein (HEX), 6-carboxyfluorescein (FAM), tetrachlorofluorescein (TET), sulfo-cyanine 3 (Cy3), sulfo-cyanine 5 (Cy5), tetramethyl rhodamine (TAMRA))) or the molecules or biochemical molecules are detectably labeled with any of the aforementioned exemplified labels; and ii) a coupling (e.g., covalent conjugation by, for example, a cysteamine molecule) to at least one plasmonic metal protrusion or at least a portion of the plurality of plasmonic metal protrusions. Yet another aspect may be directed to biochemical molecules that emit at least one surface enhanced Raman scattering (SERS) light in response to a light directed onto the plurality of plasmonic metal protrusions from an incident light source.

In conjunction with the system of the disclosure, the sample comprising, consisting essentially of, or consisting of at least one molecule or a plurality of molecules may be applied, located, coupled, conjugated, and/or hybridized to or on the at least one of or the plurality of plasmonic metal protrusions or structures (e.g. nanostructure). Another embodiment may be directed to the system of the disclosure, where each plasmonic metal protrusion or structure extending from the substrate or the surface of the surface may be, for example: cone-shaped, obelisk-shaped, pyramid-shaped, inverted funnel-shaped, wedge-shaped, cylinder-shaped, or protrusions in the shape of, for example, rings, trees, branches, rods, wires, sheets, flowers, urchins, triangles, diamonds, stars, or the like, where the plasmonic metal may be selected from, for example, gold, silver, platinum, aluminum, copper, palladium. In one embodiment, the plasmonic metal protrusion or structure may extend from the substrate or the surface of the substrate to a height of at least 10 nm (e.g., 11 nm, 20 nm, 30 nm, 40 nm, 50 nm, 60 nm, 70 nm, 80 nm, 90 nm, 100 nm, 200 nm, 300 nm, 400 nm, 500 nm); 20,000 nm or less (e.g., 19,950 nm, 10,000 nm, 8,500 nm, 1,000 nm, 750 nm, 650 nm, 550 nm, 450 nm, 350 nm, 250 nm, 150 nm, 100 nm, 50 nm, 45 nm, 35 nm, 25 nm, 15 nm); or in a range of, from, including, and/or between 10 nm-20,000 nm (e.g., 15 nm-19,500 nm, 25 nm-9,000 nm, 35 nm-8,000 nm, 45 nm-7,000 nm, 55 nm-6,000 nm, 65 nm-5,000 nm, 75 nm-4,000 nm, 85 nm-3,000 nm, 95 nm-2,000 nm, 105 nm-1,000 nm). Other embodiments provide for plasmonic metal protrusions or structures on the substrate or surface of the substrate are uniformly distributed and spaced from each other, where the width of the protrusion is at least 1 nm (e.g., 10 nm, 20 nm, 30 nm, 40 nm, 50 nm, 60 nm, 70 nm, 80 nm, 90 nm, 100 nm, 200 nm, 300 nm, 400 nm, 500 nm, 600 nm, 700 nm, 800 nm, 900 nm, 1000 nm); 2,500 nm or less (e.g., 1,500, 950 nm, 850 nm, 750 nm, 650 nm, 550 nm, 450 nm, 350 nm, 250 nm, 150 nm, 100 nm, 50 nm, 45 nm, 35 nm, 25 nm, 15 nm); or in a range of, from, including, and/or between 1 nm-2,500 nm (e.g., 10 nm-1,000 nm, 20 nm-800 nm, 30 nm-700 nm, 40 nm-600 nm, 50 nm-500 nm, 60 nm-400 nm, 70 nm-300 nm, 80 nm-200 nm, 90 nm-100 nm). Another embodiment may provide for a system of the disclosure where the plasmonic metal protrusion or structure is cone-shaped having or configured to have a height of 100 nm and a width of 250 nm, and the plasmonic metal is gold. The system of the disclosure may provide for a substrate that is glass on which the plasmonic metal protrusion or structure is deposited, where in other embodiments, Some embodiments provide for at least one molecule or a plurality of molecules in a sample, where the molecule may be directly or indirectly detectably labeled with a label, marker, biomarker, dye, or the like, or composition thereof, such as but not limited to a Raman dye (e.g., fluorophores (e.g., rhodamine X (ROX at 608 nm), rhodamine 6G, hexachlorofluorescein (HEX at 555 nm), 6-carboxyfluorescein (FAM at 520 nm), tetrachlorofluorescein (TET at 522 nm), sulfo-cyanine 3 (Cy3 at 564 nm), sulfo-cyanine 5 (Cy5 at 668 nm), tetramethyl rhodamine (TAMRA at 583 nm)).

The system of the disclosure may include or utilize a light source or an incident light source, where the light source is configured to direct a light on or onto the array or the at least one or plurality of plasmonic metal protrusions of the array, where at least one excitation frequency may result or the incident light source may be configured to direct a light having at least one excitation frequency (e.g., 2, 3, 4, 5, 6). In some embodiments, at least one molecule or a plurality of molecules or biochemical molecules may emit at least one surface enhanced Raman scattering (SERS) light in response to the light being directed onto the at least one or the plurality of plasmonic metal protrusions from the incident light source. Another embodiment of the system of the disclosure provides for a detector configured to detect at least one laser power intensity or laser excitation power and at least one Raman shift in vibrational wavenumber of the at least one Raman shift of the at least one molecule or at least one detectably labeled molecule (e.g., biomolecules, organic molecule analyte, Raman dye, label) in a SERS signal spectrum or SERS spectra. Moreover, the at least one SERS spectra may emit a signal-to-noise ratio (SNR) above or greater than a SNR predefined threshold. In some embodiments, the spectral signal-to-noise ratio values or SNR predefined threshold may be at least 1 (e.g., 10, 20, 30, 40, 50), 50 or less (e.g., 45, 35, 25, 15, 5), or in a range of, from, including, and/or between 1-50 (e.g., 4-44, 8-40, 12-34, 16-24) in, for example, the fingerprint region (e.g., 500 cm⁻¹-2400 cm⁻¹) at major peaks. In one embodiment, the SNR predefined threshold is 34.

In a further embodiment, the signal-to-noise ratio of the at least one SERS spectra may be greater than or above a SNR predefined threshold. An aspect of the embodiment provides for the SNR greater than or above a SNR predefined threshold, which may occur when a thickness of a plasmonic metal in or on each of the at least one or plurality of plasmonic metal protrusions may be at least 1 nm or 1 nm or greater (e.g., 5 nm, 10 nm, 15 nm, 20 nm, 25 nm, 30 nm, 40 nm, 45 nm, 50 nm, 55 nm, 60 nm, 65 nm, 70 nm, 75 nm, 80 nm, 85 nm, 90 nm, 100 nm, 200 nm); 200 nm or less (e.g., 250 nm, 150 nm, 125 nm, 115 nm, 98 nm, 88 nm, 78 nm, 68 nm, 58 nm, 48 nm, 38 nm, 28 nm, 18 nm, 8 nm); or in a range of, from, including, and/or between 1 nm-200 nm (e.g., 5 nm-197 nm, 7 nm-193 nm, 13 nm-183 nm, 23 nm-173 nm, 33 nm-163 nm, 43 nm-153 nm, 53 nm-153 nm, 63 nm-143 nm, 73 nm-133 nm, 83 nm-123 nm, 93 nm-113 nm). Another aspect of the embodiment provides for the SNR greater than or above a SNR predefined threshold, which may occur when the 2D material, such as for example, graphene oxide sheet or nanosheet coating, where each of the GO nanosheet coatings has a lateral size of at least 1 nm (e.g., 10 nm, 15 nm, 20 nm, 25 nm, 30 nm, 40 nm, 45 nm, 50 nm, 55 nm, 60 nm, 65 nm, 70 nm, 75 nm, 80 nm, 85 nm, 90 nm, 100 nm, 200 nm, 300 nm, 400 nm); 500 nm or less (e.g., 475 nm, 450 nm, 425 nm, 375 nm, 350 nm, 325 nm, 275 nm, 250 nm, 225 nm, 175 nm, 150 nm, 125 nm, 115 nm, 98 nm, 88 nm, 78 nm, 68 nm, 58 nm, 48 nm, 38 nm, 28 nm, 18 nm, 8 nm); or in a range of, from, including, and/or between 1 nm-500 nm (e.g., 7 nm-497 nm, 9 nm-397 nm, 13 nm-297 nm, 23 nm-295 nm, 33 nm-197 nm, 43 nm-195 nm, 53 nm-97 nm, 63 nm-95 nm, 73 nm-87 nm), or each of the GO nanosheet coatings may have a lateral size in a range of, from, including, and/or between 43 nm and 295 nm. A further aspect of the embodiment provides for the SNR greater than or above a SNR predefined threshold, which may occur when each of the GO nanosheet coatings comprising, consisting essentially of, or consisting of a thickness of at least 1 nm (e.g., 2 nm, 3 nm, 4 nm, 5 nm, 6 nm, 7 nm, 8 nm, 9 nm, 10 nm, 11 nm, 12 nm, 13 nm, 14 nm, 15 nm, 16 nm, 17 nm, 18 nm, 19 nm, 20 nm); 100 nm or less (e.g., 95 nm, 90 nm, 85 nm, 80 nm, 75 nm, 70 nm, 65 nm, 60 nm, 55 nm, 50 nm, 45 nm, 40 nm, 35 nm, 30 nm, 25 nm, 20 nm, 10 nm); or in a range of, from, including, and/or between 1 nm-100 nm (e.g., 3 nm-97 nm, 7 nm-93 nm, 13 nm-87 nm, 17 nm-83 nm, 23 nm-77 nm, 27 nm-73 nm, 33 nm-67 nm, 37 nm-63 nm, 43 nm-57 nm, 47 nm-53 nm). Yet another aspect of the embodiment provides for the SNR greater than or above a SNR predefined threshold, which may occur when composition of a Raman dye or a Raman dye is selected or chosen, wherein the composition comprises, consists essentially of, or consists of a cross-section value (e.g., Raman cross-section value) at the at least one excitation frequency at or greater than 1×10¹ Hz (e.g., 1×10² Hz, 1×10³ Hz, 1×10⁴ Hz, 1×10⁵ Hz, 1×10⁶ Hz, 1×10⁷ Hz, 1×10⁸ Hz, 1×10⁹ Hz, 1×10¹⁰ Hz, 1×10¹¹ Hz, 1×10¹² Hz, 1×10¹³ Hz, 1×10¹⁴ Hz, 2×10¹⁴ Hz, 3×10¹⁴ Hz, 4×10¹⁴ Hz, 5×10¹⁴ Hz, 1×10¹⁵ Hz, 1×10¹⁶ Hz, 1×10¹⁷ Hz, 1×10¹⁸ Hz, 1×10¹⁹ Hz, 1×10²⁰ Hz); 1×10²⁰ Hz or less (e.g., 1×10¹⁹ Hz, 1×10¹⁸ Hz, 1×10¹⁷, 1×10¹⁶ Hz, 1×10¹⁵ Hz, 5×10¹⁴ Hz, 4×10¹⁴ Hz, 3×10¹⁴ Hz, 2×10¹⁴ Hz, 1×10¹⁴ Hz, 1×10¹³ Hz, 1×10¹² Hz, 1×10¹¹ Hz, 1×10¹⁰ Hz, 1×10⁹ Hz, 1×10⁸ Hz, 1×10⁷ Hz, 1×10⁶ Hz, 1×10⁵ Hz, 1×10⁴ Hz, 1×10³ Hz, 1×10² Hz, 1×10¹ Hz); or in a range of, from, including, and/or between 1×10¹ Hz-1×10²⁰ Hz (e.g., 1×10² Hz-1×10¹⁹ Hz, 1×10³ Hz-1×10¹⁸ Hz, 1×10⁴ Hz-1×10¹⁷, 1×10⁵ Hz-1×10¹⁶ Hz, 1×10⁴ Hz-1×10¹⁵ Hz, 1×10⁵ Hz-5×10¹⁴ Hz, 1×10⁶ Hz-4×10¹⁴ Hz, 1×10⁷ Hz-3×10¹⁴ Hz, 1×10⁸ Hz-2×10¹⁴ Hz, 1×10⁹ Hz-1×10¹⁴ Hz, 1×10¹⁰ Hz-1×10¹³ Hz, 1×10¹¹ Hz, 1×10¹² Hz).

Another aspect of the embodiment provides for a processor configured to: receive, from the detector, the at least one Raman shift in vibrational wavenumber (e.g., 2, 3, 4, 5, 6) of the at least one SERS spectra (e.g., 2, 3, 4, 5, 6), the at least one laser power intensity (e.g., second, third, fourth) of the at least one SERS spectra, or any combination thereof; or data thereof (i.e., data about the at least one Raman shift in vibrational wavenumber of the at least one SERS spectra, the at least one laser power intensity of the at least one SERS spectra, or any combination thereof). Raman shift in vibrational wavenumber of SERS spectra to be detected by detector may be located at a range of, from, including, and/or between 100 cm⁻¹ to 10,000 cm¹ (e.g., 100 cm⁻¹ to 1,000 cm⁻¹; 1,250 cm⁻¹ to 3,000 cm⁻¹; 500 cm⁻¹ to 3,250 cm⁻¹; 1,500 cm⁻¹ to 5,000 cm⁻¹; 3,500 cm⁻¹ to 10,000 cm⁻¹). The power density of a Raman laser may be located at a range of, from, including, and/or between 0.01 mW/cm² to 10 mW/cm² (e.g., 0.02 mW/cm², 0.1 mW/cm², 0.5 mW/cm², 1 mW/cm², 3 mW/cm², 9 mW/cm²). A Raman laser wavelength may be located at a range of, from, including, and/or between 250 nm to 3,000 nm (e.g., 270 nm, 325 nm, 405 nm, 488 nm, 532 nm, 575 nm, 633 nm, 780 nm, 808 nm, 980 nm, 1,200 nm, 2,000 nm). Yet another aspect of the embodiment provides for a processor configured to: identify a molecule or biochemical molecules in a sample based on the at least one Raman shift in vibrational wavenumber (e.g., 2, 3, 4, 5, 6) of the at least one detectable label (e.g., Raman dye) or at least one detectable molecule (e.g., biomolecule, organic molecule analyte) SERS spectra, the at least one laser power intensity of the at least one SERS spectra, or any combination thereof.

In one embodiment of the disclosure, a system may comprise, consist essentially of, or consist of: a nanoarray, comprising, consisting essentially of, or consisting of: (1) a substrate; and (2) a plurality of plasmonic metal protrusions extending from the substrate; where the plurality of plasmonic metal protrusions has a respective plurality of graphene oxide (GO) nanosheet coatings. The system may further comprise, consist essentially of, or consist of a sample comprising, consisting essentially of, or consisting of biochemical molecules located on the plurality of plasmonic metal protrusions; where the biochemical molecules in the sample are: labeled with a Raman dye and coupled to at least a portion of the plurality of plasmonic metal protrusions. Another embodiment of the system may further provide an incident light source configured to direct a light, having at least one excitation frequency, onto the plurality of plasmonic metal protrusions; where in response to the light being directed onto the plurality of plasmonic metal protrusions from the incident light source on which a sample has been deposited thereon, the biochemical molecules of the sample may emit at least one surface enhanced Raman scattering (SERS) light. The system of the disclosure described here may also comprise, consist essentially of, or consist of a detector configured to detect at least one laser power intensity (e.g., second, third, fourth) and at least one Raman shift in vibrational wavenumber (e.g., 2, 3, 4, 5, 6) of the at least one SERS light (e.g., 2, 3, 4, 5, 6) of the at least one detectable label (e.g., Raman dye) or at least one detectable molecule (e.g., biomolecule, organic molecule analyte) in a SERS signal spectrum or SERS spectra; where a signal-to-noise ratio (SNR) of the at least one SERS spectra is above a SNR predefined threshold when: (i) a thickness of a plasmonic metal (e.g., Au, Ag, Pt, Al, Cu, Pd) in each of the plurality of plasmonic metal protrusions or nanostructure (e.g., cones, obelisks, pyramids, inverted funnels, wedges, cylinders, rings, trees, branches, rods, wires, sheets, flowers, urchins, triangles, diamonds, stars, or the like) may be in a range between 20 nm to 200 nm; (ii) a lateral size of each of the GO nanosheet coatings is in a range of, from, including, and/or between, for example, 43 nm and 295 nm and/or a thickness of each of the GO nanosheet coatings is in a range of, from, including, and/or between, for example, 1 nm-10 nm; and (iii) a composition of a Raman dye is chosen or selected to have a Raman cross-section value at or greater than 3×10¹⁴ Hz at the at least one excitation frequency (e.g., 2, 3, 4, 5, 6). Another aspect of the system may further provide a processor configured to: i) receive, from the detector, the at least one Raman shift in vibrational wavenumber of the at least one SERS spectra, the at least one laser power intensity of the at least one SERS spectra, or any combination thereof, or data about the at least one Raman shift in vibrational wavenumber of the at least one SERS spectra, the at least one laser power intensity of the at least one SERS spectra, or any combination thereof; and ii) identify the biochemical molecules in the sample based on the at least one Raman shift in vibrational wavenumber of the at least one SERS spectra, the at least one laser power intensity of the at least one SERS spectra, or any combination thereof.

In another embodiment, an array platform, e.g., nanoarray, of the disclosure may be fabricated using technologies commonly used in the art, including but not limited to laser interference lithography (LIL) followed by physical vapor deposition (PVD) of a metal (e.g., gold (Au), silver (Ag), platinum (Pt), aluminum (Al), copper (Cu), palladium (Pd)) and then electrostatically coated with graphene oxide (GO) nanosheets. In a further embodiment, a high SERS signal enhancement due to an EM effect with a minimal signal variation may be achieved by creating a homogeneous and uniform noble-metal nanostructured array and identifying an excitation wavelength having an optimal cross-section overlap with the metal nanostructures. Non-limiting examples of excitation wavelengths include 520 nm, 522 nm, 555 nm, 564 nm, 583 nm, 608 nm, 668 nm, that are useful for the detection of molecules of interest, (e.g., biochemical molecules), labeled molecules of interest Raman dyes, such as but not limited to fluorophores (e.g., rhodamine X (ROX at 608 nm), rhodamine 6G, hexachlorofluorescein (HEX at 555 nm), 6-carboxyfluorescein (FAM at 520 nm), tetrachlorofluorescein (TET at 522 nm), sulfo-cyanine 3 (Cy3 at 564 nm), sulfo-cyanine 5 (Cy5 at 668 nm), tetramethyl rhodamine (TAMRA at 583 nm)) may be selected to have a strong interaction with the excitation wavelength, thereby allowing for maximum plasmonic coupling between the excitation wavelength, Raman dye, and metal (e.g., Au, Ag, Pt, Al, Cu, Pd) nanostructure.

One embodiment provides for an enhanced Raman signal of the dye by optimizing the energy levels of graphene oxide, which occurs by modification of an array (e.g., nanoarray, microarray) surface with graphene oxide and subsequent partial reduction of the GO surface. As a result, the charge transfer processes are strengthened between partially reduced graphene oxide (rGO) and adsorbed molecules. Consequently, the synergistic effect from, for example, a homogeneous gold nanoarray and reduced graphene oxide, the exemplary graphene-Au hybrid nanoarray disclosed here enabled a homogeneous dual-enhanced Raman scattering by combining both electromagnetic mechanism- and chemical mechanism-based enhancements.

Another embodiment may be directed to a biosensing system for use as a sensitive and selective nucleic acid sensor with a high signal-to-noise ratio for characterizing, for example, neural stem cell differentiation. An advantage of the unique surface properties of rGO allows target-specific detection of cell-derived biomolecules, such as DNA and RNA, in complex biological fluids, e.g., mucus, cell membranes, the cytoskeleton, and blood. In some aspects, rGO can selectively bind Raman dye-labeled nucleic acid probes near to the plasmonic field (<10 nm), which can be characterized by strong Raman signals on the dual-enhanced SERS platform of the disclosure via noncovalent forces such as π-π interactions. Without being bound by theory, when target nucleic acid biomolecules (e.g., DNA, RNA) with complementary sequences derived from cells are in the presence of adsorbed Raman dye-labeled DNA, Watson-Crick base pairing leads to helix formation and detachment of Raman dye-labeled nucleic acid probes, thereby resulting in a change of Raman signals. This phenomenon provides, in another embodiment, a system for quantifying RNA concentrations and gene expression levels of biomarkers (e.g., neuron biomarker, TuJ1; astrocyte biomarker, GFAP). A further embodiment provides a dual-enhanced SERS platform or system for sensing cell-derived biomolecules, which allows for large scale and homogeneous detection of genes and small molecules with high sensitivity, good signal-to-noise ratio, and the ability to quantify absolute biomarker concentrations.

Obtaining reliable and reproducible SERS signals are essential for the precise and quantitative analysis of biomolecular interactions. It has been well-known that homogeneity of the plasmonic nanostructure is crucial for obtaining consistent enhancement of Raman signals with minimal variation.

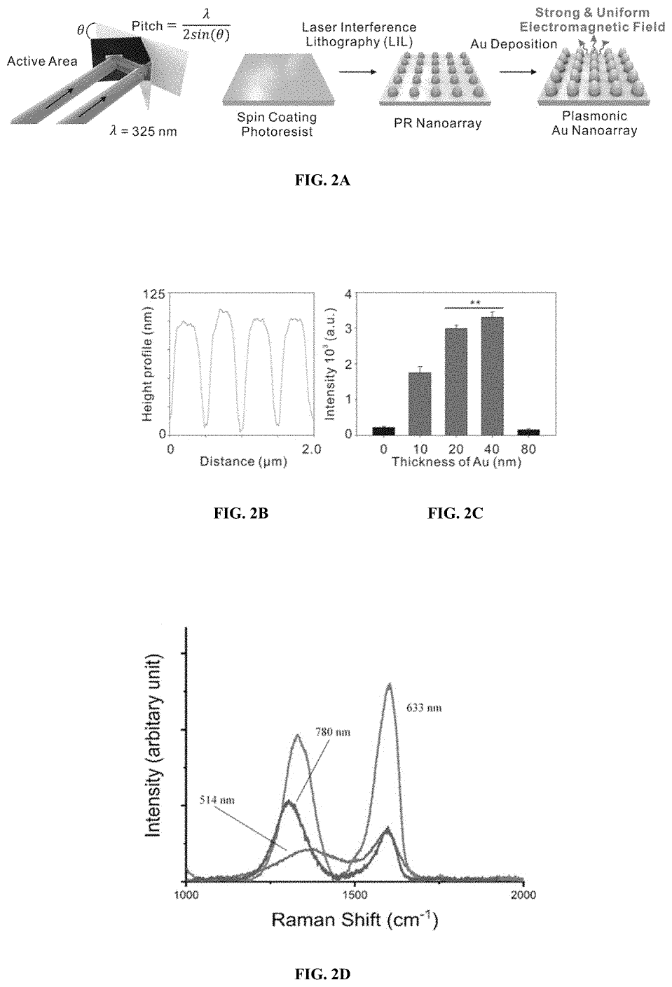

Other embodiments of the disclosure provide a uniform homogeneous plasmonic metal protrusion (e.g., cone-shaped Au) nanoarray (FIG. 2A) designed to obtain reliable and reproducible SERS signals, which are critical for the precise and quantitative analysis of biomolecular interactions. In order to attain consistent Raman signal enhancement with minimal variation, homogeneous plasmonic nanostructure was essential. Some embodiments of the disclosure provide for a highly uniform homogeneous plasmonic metal protrusion nanoarray exemplified in FIG. 2A, which, for example, has a cone-shaped Au nanoarray. The anisotropic nanocone shape may be spaced at a subwavelength distance (<<λ) in order to achieve strong localized surface plasmon resonance (LSPR) coupling through an increased cross-section with the excitation laser. For example, a large-scale (e.g., 1×1 cm²) homogeneous photoresist (PR) array (e.g., nanoarray, microarray) may be prepared using laser interference lithography on a substrate (e.g., glass, silicon). A uniform hologram may be obtained as a result of the homogeneously developed PR nanostructures over the surface of, for example, glass. A homogeneous plasmonic metal (e.g., Au, Ag, Pt, Al, Cu, Pd) may be deposited on a substrate to act as a plasmonic layer via physical vapor deposition (PVD) on the photoresist nanoarray. Both scanning electron microscopy (SEM) and atomic force microscopy (AFM) may be used to characterize the surface of the Au nanoarray described here (FIG. 1B). The width and gap of a metal nanoarray of the disclosure may be, for example, 100 nm-2500 nm (e.g., 10 nm-2000 nm, 50 nm-1000 nm, 100 nm-500 nm, 125 nm-250) while the height may be, for example, 1 nm-200 nm (e.g., 5 nm-180 nm, 10 nm-170 nm, 20 nm-150 nm, 30 nm-125 nm, 40 nm-100 nm, 50 nm-80 nm) (FIG. 2B). As shown in FIG. 2C, Raman signal enhancement may occur after deposition of a 10 nm layer of a metal (e.g., Au) while saturation occurs after deposition of the layer at a distance at or between 20 nm to 40 nm. However, after depositing an 80 nm thick layer of the metal, saturation levels diminished. Accordingly, the thickness of the metal layer correlates to the intensity. Electromagnetic simulations may be conducted using three different incident light sources (e.g., 514, 633, 780 nm, or any wavelength sufficient to detect a Raman dye or excitation), which are representative excitation wavelengths that typically provide strong localized surface plasmon resonance (LSPR) in such Au metal nanostructures. Some aspects provide the dimensions and shapes of the structures on a substrate of the disclosure, which may include but are not limited to, cones, obelisks, pyramids, inverted funnels, wedges, cylinders, rings, trees, branches, rods, wires, sheets, flowers, urchins, triangles, diamonds, stars, or the like. These structures may also be of a size, such as for example, a nanostructure, a microstructure, or the like. Using finite difference time-domain (FDTD) simulations, a nanocone plasmonic array closely representing an actual SERS substrate may be created. In FDTD, Raman enhancement may be proportional to the fourth power of local electric field enhancement. As such, the local electric field enhancement [|E|/|E₀|, the ratio of near-field (|E|) and the incident field (|E₀|)], may be calculated and plotted (FIG. 2D). Electric field distribution images illustrate that when, for example, Au metal nanoarrays are exposed to incident light (λ: 514, 633, and 780 nm), the local electric field enhancement slightly increased for both the 514 nm and 780 nm wavelengths; however, the most significant signal enhancement on a scale of 0 to 8 was found to occur at a wavelength of 633 nm, owing to the unique anisotropic nanocone structure of the plasmonic noble-metal (here, Au). Incident light with 633 nm wavelength may be selected and utilized for inducing Raman signal enhancement.

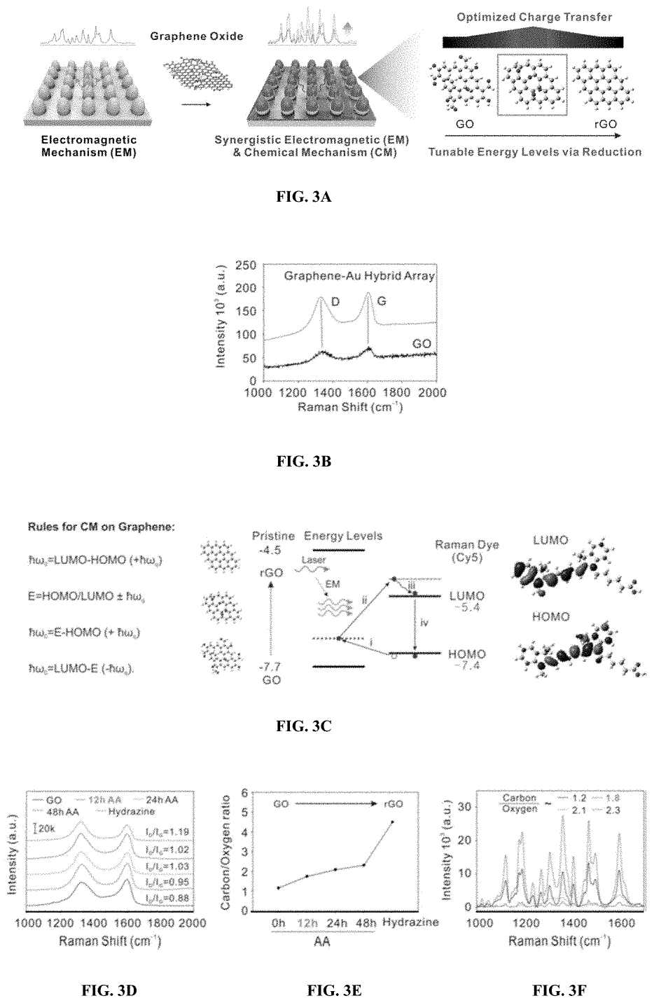

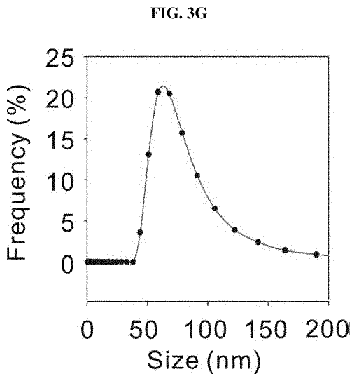

Further embodiments may be directed to platforms or systems for optimizing energy levels of a two-dimensional (2D) material, e.g., graphene oxide (GO), to maximize Raman signal enhancement via a chemical mechanism on a plasmonic metal (e.g., Au, Ag, Pt, Al, Cu, Pd) nanoarray. Two-dimensional graphene oxide nanosheets synthesized by a modified Hummers' method may provide synergistically improved Raman signals by the CM-based enhancement in addition to the EM-based enhancement obtained from the plasmonic Au nanoarray (FIG. 3A). The functionalization of an array (e.g., nanoarray) surface with physiochemically defined two-dimensional materials (e.g., graphene oxide) can enhance the Raman signal by optimizing the charge transfer processes through tuning the energy levels of GO. Graphene can quench spectral fluorescent background effectively, provide binding moieties for analytes, and increase resistance to environmental effects, which makes it an attractive material for surface functionalization. To obtain consistent Raman signal enhancement, the size of exemplary GO nanosheets may be adjusted by utilizing ultrasonication followed by filtration (e.g., nanofiltration; average pore size of filter: 0.2 μm in diameter) to optimize the coverage of the metal (e.g., Au, Ag, Pt, Al, Cu, Pd) nanoarray surface. Prepared GO nanosheets may be characterized by transmission electron microscopy (TEM). The lateral size distribution of the GO sheets may be greater than or equal to 1 nm (e.g., 5 nm, 10 nm, 20 nm, 30 nm, 40 nm, 50 nm, 60 nm, 70 nm, 80 nm, 90 nm, 100 nm, 200 nm, 300 nm, 400 nm, 500 nm, 1000 nm, 5000 nm, 10,000 nm, 15,000 nm), 20,000 nm or less (e.g., 19,500 nm, 18,500 nm, 17,500 nm, 16,500 nm, 15,500 nm, 14,500 nm, 10,500 nm, 5,500 nm, 2,500 nm, 550 nm, 450 nm, 350 nm, 250 nm, 150 nm, 50 nm, 45 nm, 35 nm, 25 nm, 15 nm), or range from 1 nm-20,000 nm (e.g., 1 nm-500 nm, 10 nm-500 nm, 5 nm-20,000 nm, 20 nm-450 nm, 25 nm-400 nm, 35 nm-350 nm, 45 nm-300 nm, 55 nm-250 nm, 65 nm-150 nm). For example, the size distribution may be in a range of 43 nm to 295 nm, such as 43.82 nm to 295.3 nm, with an average size of 77.27 nm as analyzed by dynamic light scattering (DLS) analysis (FIG. 3G).

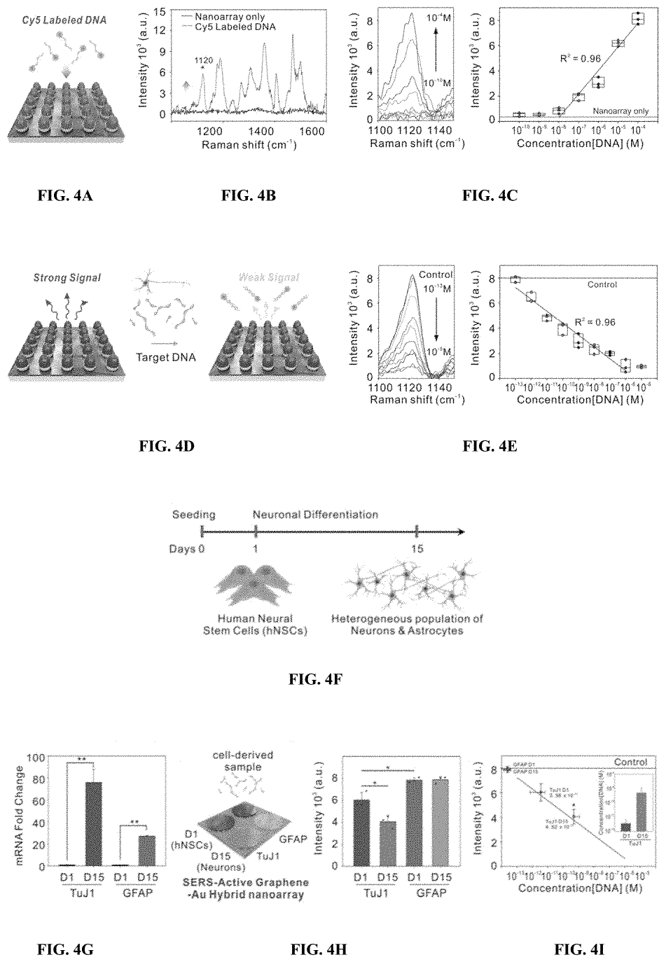

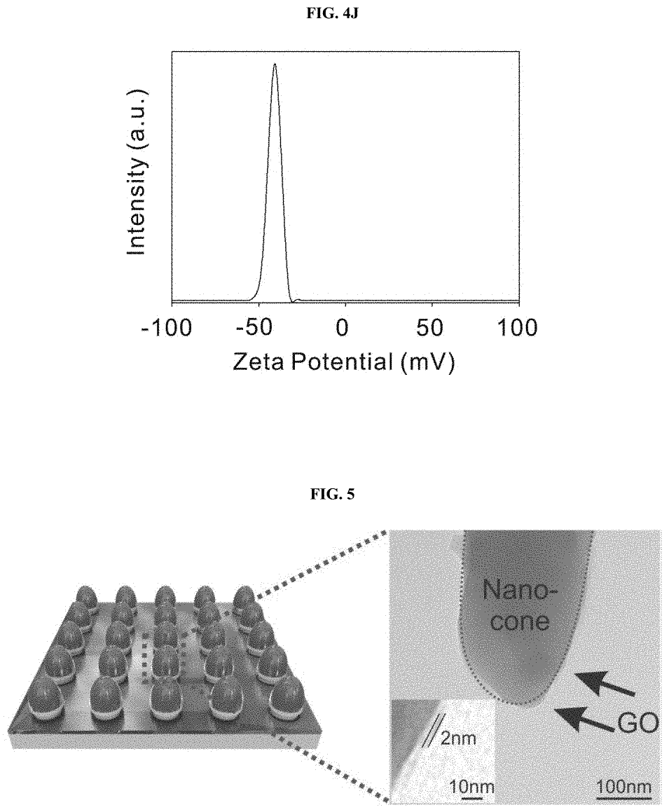

Another embodiment may be directed to a platform or system of a surface of a metal array, such as an Au nanoarray, functionalized with GO on its surface through electrostatic interactions by utilizing a chemical linker, e.g., cysteamine hydrochloride (C₂H₇NS.HCl), thiol carboxylics, thiol polyethylene glycols, carboxylic amines, thiol amines, amine silanes, and others. At least one thin layer (e.g., 1-5 layers) or a plurality of layers of GO at a thickness ranging in nanometers (e.g., 0.05 nm-5 nm, 0.1 nm-3.5 nm, 1 nm-2 nm), for example, 1-2 nm with 1-3 layers, where the structure of GO may be well-preserved based on TEM characterizations (FIG. 4-1; FIG. 5). Graphene oxide-Au hybrid nanoarrays having, for example, Raman transition bands, of the distinct D (1350 cm⁻¹) and G (1600 cm⁻¹) bands of GO, may be a result of the unique physicochemical structure of atomically thin layered GO (FIG. 3B). In order to analyze defect density (i.e., total defect/size) of materials, including but not limited to carbon materials such as GO, the D and G bands in the Raman scattering. Moreover, in some embodiments, a homogeneous distribution of Raman signals of GO (distinct G band, 1600 cm⁻¹, of GO) may occur over a large scan area ranging from 10×10 spots (per 10×10 μm²)-200×200 spots per 200×200 μm² (e.g., 80×80 spots per 80×80 μm²) from the graphene-Au hybrid nanoarray with 8% relative standard deviation (RSD), owing to a surface-enhanced Raman scattering effect caused by the disclosed platform or system, e.g., a homogeneous GO-coated plasmonic Au nanoarray.

Other embodiments may be directed to a system of the disclosure and the combined effect between chemical mechanism (CM) from 2D material (e.g., graphene oxide nanosheets) and electromagnetic mechanisms (EM) from a plasmonic metal (e.g., Au) array. A detectable label, marker, or biomarker all used interchangeably herein, such as a Raman dye (e.g., fluorophores (e.g., rhodamine X (ROX at 608 nm), rhodamine 6G, hexachlorofluorescein (HEX at 555 nm), 6-carboxyfluorescein (FAM at 520 nm), tetrachlorofluorescein (TET at 522 nm), sulfo-cyanine 3 (Cy3 at 564 nm), sulfo-cyanine 5 (Cy5 at 668 nm), tetramethyl rhodamine (TAMRA at 583 nm), where the Raman dye may be Cyanine 5 (Cy5) may be used to achieve a high Raman cross-section with a preselected laser wavelength (e.g., 633 nm), which provides electromagnetic enhancement based on FDTD simulation results. FIG. 3C illustrates the CM-based enhancement rules resulting from other 2D graphene-analogs

hω0=LUMO−HOMO(+hωq) (1)

In the equation (1), h is the Plank constant; LUMO is the lowest unoccupied molecular orbital; HOMO is the highest occupied molecular orbital; ω₀ is the frequency of incident Raman laser; and ω_{q} is the frequency of scattered photons. In terms of enhanced Raman spectroscopy on graphene and its derivatives, which is also termed as “GERS”, there are additional equations that define the efficiency of chemical enhancement based on the molecular orbitals of the Raman dye and the energy levels of graphene (E_{G}, FIG. 3C). To optimize the CM-based enhancement of the Cy5 dye on the graphene-Au hybrid nanoarray of the disclosure, density functional theory (DFT) calculations using a graphene oxide macromolecule mimic. On the basis of the simulation results, as well as the basic principles of GERS, energy levels of pristine graphene, fully reduced GO, and non-reduced GO, fell outside the optimal range of GERS for the Cy5 dye. However, by modulating the energy levels of partially reduced GO (rGO), a strong charge-transfer (e.g., greater than 3% changes of positive/negative charges from the shift, e.g., GO and rGO) and optimal GERS effect could be achieved. Should the energy level of rGO be too high (e.g., greater than 3.0 eV higher than the LUMO of organic molecules or analytes or dyes), the charge transfer can be impeded. Contrarily, if the energy level of rGO is too low (e.g., greater than 3.0 eV lower than the HOMO of organic molecules or analytes or dyes), the laser excitation may also not necessarily lead to sufficient charge transfer to Cy5 (FIG. 3C).

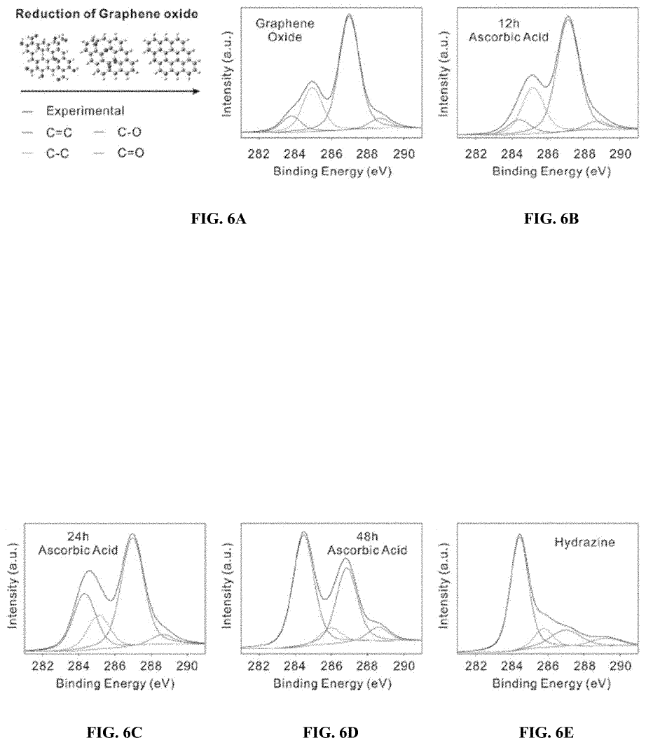

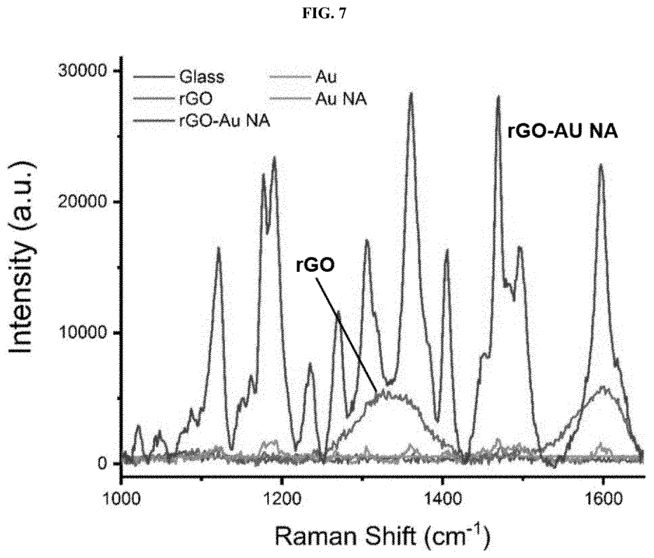

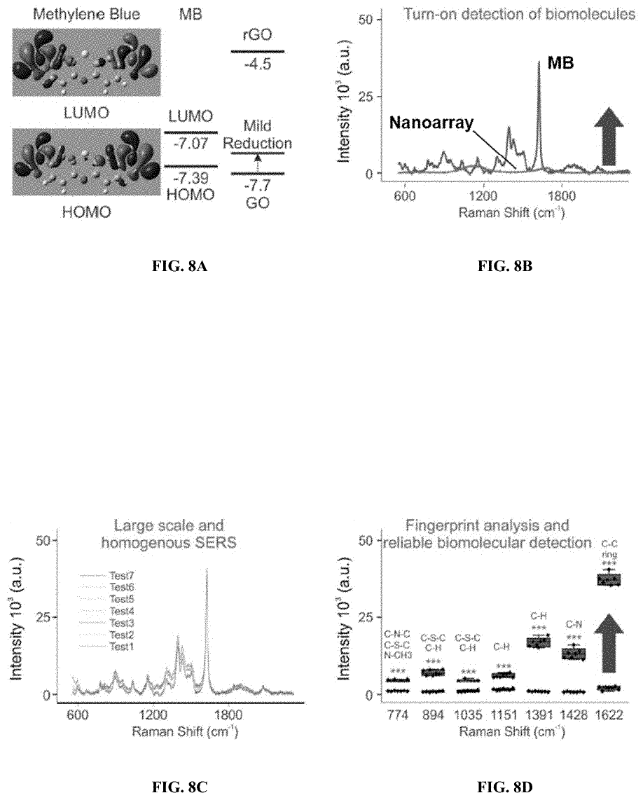

A further embodiment provides an optimized energy level of graphene oxide for stimulating the charge transfer from GO nanosheets to the Raman active reporter (e.g., Cy5), which may occur by chemically reducing GO on the graphene oxide-Au hybrid nanoarray of the disclosure. The reduction rate of GO to rGO may be verified by comparing the Raman intensity ratio between the D and G bands (I_{D}/I_{G}) after the reduction process. FIG. 3D illustrates an I_{D}/I_{G} ratio that increases from 0.88 (lowest curve, GO) to 1.19 (highest curve, hydrazine; FIG. 6E) as GO reduces to rGO (FIG. 6A) with intervening curves represented by 12 hours (FIG. 6B), 24 hours (FIG. 6C), 48 hours (FIG. 6D) under ascorbic acid (AA) conditions, respectively as the intensity (y-axis) increases in arbitrary units (a.u.). To further quantify reduction levels, X-ray photoelectron spectroscopy (XPS) may be performed (FIG. 3E; FIG. 6). Consistent with results from the Raman experiment, the calculated carbon to oxygen ratios obtained from XPS spectra may increase in a stepwise manner from a carbon to oxygen ratio of 1.2 to 4.49 as GO reduces to rGO, which may be accompanied by the removal of electron-withdrawing groups, such as but not limited to, hydroxyl, carboxylic, and epoxy groups. As a result, energy levels (e.g., LUMO) of rGO can rise to a higher energy state and modulate charge transfer between the substrate and the adsorbate. More specifically, compared to untreated GO (carbon to oxygen ratio: 1.2), the rGO with carbon to oxygen ratio of 1.8 was found to have a remarkable enhancement of the Cy5 Raman signal, suggesting an optimal CM-based enhancement through the modulation of energy levels of GO. In contrast, when GO was highly reduced to the rGO with carbon to oxygen ratio of 2.1 and 2.3, the SERS signals of Cy5 were diminished, which reflected the trend predicted in the simulation diagram and support the dual-enhanced SERS mechanism (FIG. 3F; FIG. 7). The graphene-Au hybrid of the disclosure demonstrates higher homogeneity of SERS signals as compared to GO/Au nanoparticle (NP) core-shell composites and GO/Au nanorod (NR) core-shell composites, where the graphene-Au hybrid has an intensity (10³ a.u.) of more than 12 and E/E₀ of greater than 1.5, while the other GO/Au hybrids (i.e., NP, NR) express an intensity of less than 12 and an E/E₀ of less than 1.5. The graphene-Au hybrid of the disclosure demonstrated an advantageously higher homogeneity of SERS signals over either nanoparticle core-shell composites or nanorod core-shell composites. A fingerprint analysis of a different type of molecule (e.g., methylene blue; FIG. 8B) demonstrated energy levels aligned with those observed with the graphene-Au hybrid of the disclosure (FIG. 8A). One embodiment provides for an optimized reduction condition of GO combined with the EM-based enhancement of the Au nanoarray of the disclosure, which results in a sensitive, yet reproducible, SERS platform for the detection of bio/chemical molecules. FIG. 8C shows repeated tests using large scale and homogeneous SERS. FIG. 8D illustrates a fingerprint analysis and reliable biomolecular detection.

An exemplary use of the graphene-Au hybrid nanoarray-based biosensors of the disclosure includes monitoring advanced cellular processes, such as but not limited to, stem cell neuronal differentiation. The SERS nanoarray of the disclosure may be used for nucleic acid (e.g., DNA, RNA) detection. Among various bio/chemical molecules, nucleic acids play a fundamental role in investigating a variety of biological phenomena, including disease progression and stem cell differentiation. Accordingly, quantification of specific nucleic acids allows for monitoring such biological processes. Disadvantages or critical challenges of conventional tools that the graphene-Au hybrid nanoarray of the disclosure overcomes include, for example, expensive instrumentation, unsatisfactory signal-to-noise ratio, and a limited ability to quantify absolute RNA concentrations for gene detection in lysed cells.

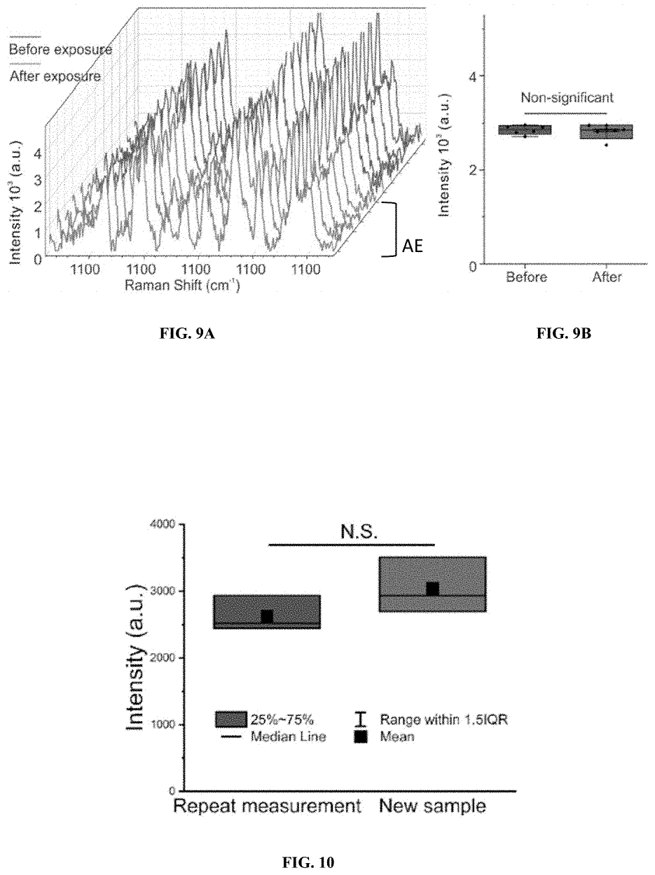

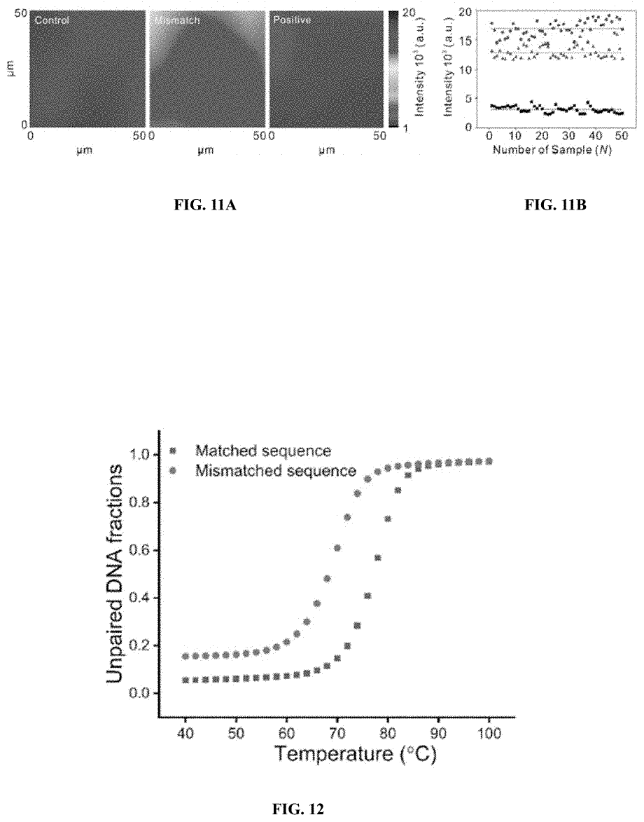

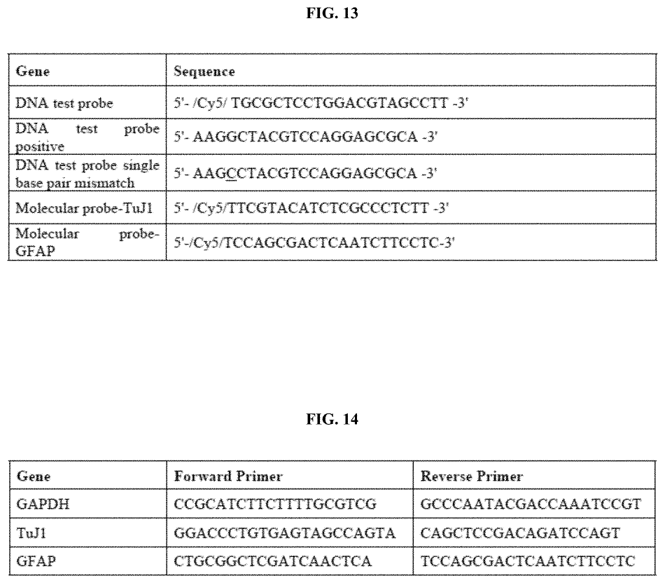

In yet another embodiment a system or platform may provide for improved nucleic acid gene detection, a Raman active reporter dye (e.g., Cy5) may be conjugated onto a nucleic acid probe (e.g., DNA) to generate a Raman reporter for specific DNA sequences. The surface of the graphene-Au hybrid nanoarray may be functionalized with the Raman active reporter dye to form a highly sensitive nucleic acid sensor. Probe nucleic acid DNA strands may be adsorbed onto the surface of the graphene-Au hybrid nanoarray through π-π stacking. For example, a synthetic probe DNA with 21 base pairs may be designed and used as a target molecule. (FIG. 4A; FIG. 13). Monitoring the DNA based on distinctive peaks of Cy5 [1120 cm⁻¹ (C—H vibration mode of Cy5), 1190 cm⁻¹, 1280 cm⁻¹, 1361 cm⁻¹, 1405 cm⁻¹, 1468 cm⁻¹, and 1593 cm⁻¹] after absorption of the DNA probe (FIG. 4B). Among the varying peaks, the Raman transition band at 1120 cm⁻¹ may be selected for quantitative measurements to avoid the background signal (i.e., D and G bands) from an aromatic ring structure of the rGO surface and due to its high signal stability (FIG. 9A; FIG. 10). On the basis of the 1120 cm⁻¹ Raman peak, a concentration dependent detection of the DNA probe in a wide range from 1 nM to 100 μM with linearity R²=0.96 (FIG. 4C). Also, while using a low concentration (1 μM) of DNA, a high signal-to-noise ratio (SNR) of 34 was calculated for the system, based on an equation,

SNR=S/D (2)

where S stands for the average of peak intensities at 1120 cm⁻¹ and D stands for the standard deviation of signals with independent experimental number of 6, which represents an improvement compared to conventional Raman- or fluorescence-gene analysis tools (FIG. 9B). After demonstrating “turn-on” detection of Cy5-labeled DNA in a sensitive manner, selective gene detection may be performed by absorbing Cy5-labeled DNA probe with complementary sequences to the target DNA (FIG. 4D; FIG. 14), for example, via the π-π interactions. Reduced GO (rGO) can bind Raman dye-labeled nucleic acid probes near to the plasmonic field (<10 nm), which may be characterized by strong Raman signals on the dual-enhanced SERS system or platform of the disclosure. When target DNA or RNA with complementary sequences derived from cells are in the presence of the adsorbed Raman dye-labeled DNA, Watson-Crick base pairing leads to helix formation and the detachment of Raman dye-labeled nucleic acid probes, resulting in a significant change of Raman signals.

Because of DNA base pairing specificity, the change of Raman signal from the complementary DNA sequence may be significantly higher than the single-base mismatched DNA sequence (10 nM) (FIG. 11A-FIG. 11B; FIG. 12; FIG. 14). The stability of the graphene-Au hybrid nanoarray sensor system of the disclosure by identifying non-significant changes of the SERS signals from Cy5 over the time periods equivalent to the assay time. A large-scale homogeneous graphene-Au hybrid nanoarray of the disclosure provides high sensitivity, selectivity, stability, and the ability to quantify RNA concentrations as compared to conventional gene analysis tools. In another embodiment, the SERS-based platform of the disclosure may be applied to versatile biosensing applications in view of graphene-based reversible absorption and target-specific detachment of dye-labeled nucleic acids. Other applications may include, but are not limited to, aptamer-based live-cell protein and small molecule detection. The large-scale homogeneous graphene-Au hybrid nanoarray SERS-based platform of the disclosure is advantageous over other specialized gene analysis techniques such as PCR.

Cell-derived DNA samples may be used to test sensitivity and selectivity of the graphene-Au hybrid SERS array of the disclosure. The system described here may detect the conversion of human neural stem cells (hNSCs) into neurons. Neuronal differentiation of hNSCs (4.0×10⁴ cells/cm²) may be induced by removing the fibroblast growth factor (FGF basic) from proliferation media. Neuronal differentiation of hNSCs may be confirmed through immunocytochemistry staining of Nestin and neuron-specific class III β-tubulin (TuJ1), which are representative markers of hNSCs and differentiated neurons, respectively. Undifferentiated hNSCs (D1) show clear Nestin expression, and the cells that have undergone neuronal differentiation (D15) show clear TuJ1 expression, which is consistent with gene analysis results from PCR experiments after the conversion of cellular mRNA into DNA followed by PCR-based amplification (FIG. 4H). The graphene-Au hybrid SERS nanoarray may be functionalized with DNA probes targeting TuJ1 and indicate the same trend by performing gene detection of cell-derived DNA samples (FIG. 4I). Consistent with the immunostaining results, significantly less changes of SERS signal from differentiated stem cells compared to undifferentiated stem cells compared to the TuJ1 detection experiment may be observed, suggesting a favored neuronal differentiation of the stem cell line. Furthermore, based on the calibration curve in FIG. 4E, the SERS-based sensing platform of the disclosure may be used to quantify the absolute concentrations of DNA, which are essential for a complete understanding toward genome and transcriptome (FIG. 4I). In an exemplary use of the system of the disclosure, FIG. 4F presents a timeline of neuronal cell differentiation from a human neural stem cells (hNSCs) that after 15 days post seeding results in a heterogeneous population of neurons and astrocytes, where the biological molecules are hNSCs. FIG. 4G illustrates RT-PCR experiments characterizes the neuronal differentiation of hNSCs after amplification of the DNA using PCR comparing TuJ1 and GFAP on Day 1 (D1) versus Day 15 (D15) demonstrating statistically significance (**). FIG. 4H shows graphene-Au hybrid nanoarray-based sensing of labels TuJ1 and GFAP obtained from hNSCs before (D1) and after neuronal differentiation (D15). In one embodiment, the disclosed SERS-based sensing platform may increase Tuj1 RNA level by 2 orders of magnitude, which matches the PCR results (FIG. 4J). The graphene-Au hybrid SERS array may be functionalized with probe DNA targeting glial fibrillary acidic protein (GFAP), which is mostly expressed in glial cells of the central nervous system such as, for example, astrocytes. Although conventional PCR techniques can detect a relative change in gene expression of GFAP at Day 15 (D15) through gene amplification shown through immunostaining, there are no GFAP-positive cells. Yet, with the graphene-Au hybrid array SERS system disclosed here, the total copy number of GFAP RNA at Day 15 is extremely low (FIG. 4J). Even though PCR can be an extremely sensitive method to detect relative changes in gene expression, it does not always give results representative of the differentiation state of cells. Yet, the graphene-Au hybrid SERS array system of the disclosure, on the other hand, provides a quantitative analysis of total gene expression levels. In addition to the embodiment for detecting two genes, multigene detection could facilely be achieved on a single chip by simply spotting or depositing different probe nucleic acids (e.g., DNAs) in a spatially resolved manner. Accordingly, the hybrid SERS array of the disclosure provides both sensitive and quantitative analyses of multiple genes toward monitoring cellular behaviors.

In one embodiment of the disclosure, a SERS-based biosensing system or platform comprises, consists essentially of, or consists of a large-scale, homogeneous 2D material-metal hybrid array. Some embodiments provide a graphene-Au hybrid nanoarray which exhibits synergistic enhancement of Raman signal from both EM and CM. A further embodiment may be directed to the graphene-Au hybrid nanoarray of the disclosure which may be used as a highly sensitive and selective biosensing system for monitoring cells, such as but not limited to, neuronal differentiation of stem cells. In some aspects, Raman dye-labeled synthetic probe DNA oligonucleotides may be conjugated onto the nanoarray to detect the biomarker/complementary DNA strands (TuJ1, a neuronal lineage marker).

Another embodiment may provide for a SERS-based biosensing system or platform of the disclosure as a highly sensitive and reproducible sensing system targeting specific stem cell differentiation biomarkers with the ability to quantify the gene concentrations (e.g., TuJ1 mRNA). The biosensing system has unique features, including but not limited to, a synergistic effect from the plasmonic coupling between the laser, dye, homogeneous Au nanoarray, as well as the modulation of surface energy levels from graphene nanostructures. The selectivity of the graphene-Au hybrid SERS nanoarray may be verified with real cell-derived gene expression analysis. In yet another embodiment, the graphene-Au hybrid SERS nanoarray of the disclosure may be modified with an additional probe DNA strand (e.g., glial fibrillary acidic protein (GFAP), an astrocyte lineage marker). By integrating combinatorial microarrays of probe DNAs using advanced spotting techniques, multiplexed gene detection can also be achieved in a single substrate for high throughput gene analysis. A further embodiment capitalizes on the versatility of oligonucleotide-based probes (e.g., aptamer) and the graphene-Au hybrid SERS nanoarray of the disclosure may be used to detect other bio/chemical molecules (e.g., small molecule, protein, and the like) as well. Therefore, while the gene detection system may not be directly applied to monitoring biomolecular interactions in the cellular context, in one embodiment, the hybrid SERS nanoarray system of the disclosure may detect in a non-destructive manner. The graphene-Au hybrid SERS nanoarray system may be used for high-quality and high throughput bio/chemical molecule screening assays and also enable an understanding of cellular phenomena such as disease progression and stem cell differentiation, thus leading to more effective therapies.

### Method

One embodiment of the disclosure provides a method, comprising, consisting essentially of, or consisting of:

disposing or applying a sample onto at least one of or a plurality of plasmonic metal (e.g., Au, Ag, Pt, Al, Cu, Pd) protrusions (e.g., nanostructures) extending from a substrate (e.g., glass, silicon); where the plurality of plasmonic metal protrusions has a respective at least one or a plurality of 2D materials (e.g., nanomaterial, graphene oxide (GO), graphene, graphane, graphyne, 2D boron nitride, borophene, germanene, silicene, Si₂BN, stanene, plumbene, phosphorene, antimonene, bismuthene, platinum, palladium, rhodium, 2D alloys, 2D crystals) sheet (e.g., nanosheet) coatings or layers (e.g., 1 layer, 2 layers, 3 layers, 4 layers, 5 layers); where the sample comprises, consists essentially of, or consists of at least one molecule or a plurality of molecules (e.g., biochemical molecule; biomolecule; small molecule; drug; nucleic acids (e.g., DNA, DNA strand, DNA sequence, cDNA, RNA, RNA strand, DNA sequence, mRNA, miRNA); cell (e.g., stem cell, neural stem cell, differentiating cell, cell-derived vesicle); proteins; metabolites, pathogen, antigen, virus or viral particle: influenza virus (e.g., influenza A, zoonotic influenza, influenza B), respiratory syncytial virus, parainfluenza virus, adenovirus, rhinovirus, metapneumovirus, human metapneumovirus and endemic human coronaviruses (e.g., HKU1, OC43, NL63, 229E), enterovirus (e.g., EVD68), and coronavirus (e.g., MERS-CoV, SARS-CoV, SARS-CoV-2 or 2019-nCoV)) that are coupled to at least a portion of the at least one or a plurality of plasmonic metal protrusions or structure having a shape selected from, for example, cones, obelisks, pyramids, inverted funnels, wedges, cylinders, rings, trees, branches, rods, wires, sheets, flowers, urchins, triangles, diamonds, stars, or the like;

labeling or detectably labeling the biochemical molecules in the sample with a label, marker, biomarker, dye, or the like, or composition thereof, such as but not limited to a Raman dye (e.g., fluorophores (e.g., rhodamine X (ROX at 608 nm), rhodamine 6G, hexachlorofluorescein (HEX at 555 nm), 6-carboxyfluorescein (FAM at 520 nm), tetrachlorofluorescein (TET at 522 nm), sulfo-cyanine 3 (Cy3 at 564 nm), sulfo-cyanine 5 (Cy5 at 668 nm), tetramethyl rhodamine (TAMRA at 583 nm));

illuminating the at least one or a plurality of plasmonic metal protrusions with a light directed from an incident light source having at least one excitation frequency (e.g., 2, 3, 4, 5, 6); where the biochemical molecules of the sample deposited on or applied to the at least one or a plurality of plasmonic metal protrusions emit at least one surface enhanced Raman scattering (SERS) light (e.g., 2, 3, 4, 5, 6), in response to the light being directed onto the at least one or a plurality of plasmonic metal protrusions from the incident light source; Raman shift in vibrational wavenumber of SERS spectra to be detected by detector may be located at a range of, from, including, and/or between 100 cm⁻¹ to 10,000 cm⁻¹ (e.g., 100 cm⁻¹ to 1,000 cm⁻¹; 1,250 cm⁻¹ to 3,000 cm⁻¹; 500 cm⁻¹ to 3,250 cm⁻¹; 1,500 cm⁻¹ to 5,000 cm⁻¹; 3,500 cm⁻¹ to 10,000 cm⁻¹). The power density of a Raman laser may be located at a range of, from, including, and/or between 0.01 mW/cm² to 10 mW/cm² (e.g., 0.02 mW/cm², 0.1 mW/cm², 0.5 mW/cm², 1 mW/cm², 3 mW/cm², 9 mW/cm²). A Raman laser wavelength may be located at a range of, from, including, and/or between 250 nm to 3,000 nm (e.g., 270 nm, 325 nm, 405 nm, 488 nm, 532 nm, 575 nm, 633 nm, 780 nm, 808 nm, 980 nm, 1200 nm, 2000 nm).;

detecting, by a detector, at least one laser power intensity (e.g., second, third, fourth) and at least one Raman shift in vibrational wavenumber (e.g., 2, 3, 4, 5, 6) of the at least one detectable label (e.g., Raman dye) or at least one detectable molecule (e.g., biomolecule, organic molecule analyte) in a SERS signal spectrum or SERS spectra (e.g., 2, 3, 4, 5, 6);

increasing a signal-to-noise ratio (SNR) of the at least one SERS spectra above a SNR predefined threshold (e.g., at least 1 (e.g., 10, 20, 30, 40, 50); 50 or less (e.g., 45, 35, 25, 15, 5); or in a range of, from, including, and/or between 1-50 (e.g., 4-44, 8-40, 12-34, 16-24)) by:

varying a thickness of a plasmonic metal in the plurality of plasmonic metal protrusions to be at least 5 nm or 5 nm or greater (e.g., 10 nm, 15 nm, 20 nm, 25 nm, 30 nm, 40 nm, 45 nm, 50 nm, 55 nm, 60 nm, 65 nm, 70 nm, 75 nm, 80 nm, 85 nm, 90 nm, 100 nm, 200 nm); 200 nm or less (e.g., 250 nm, 150 nm, 125 nm, 115 nm, 98 nm, 88 nm, 78 nm, 68 nm, 58 nm, 48 nm, 38 nm, 28 nm, 18 nm, 8 nm); or in a range of, from, including, and/or between 5 nm-200 nm (e.g., 7 nm-193 nm, 13 nm-183 nm, 23 nm-173 nm, 33 nm-163 nm, 43 nm-153 nm, 53 nm-153 nm, 63 nm-143 nm, 73 nm-133 nm, 83 nm-123 nm, 93 nm-113 nm); or in a range of, from, including, and/or between 20 nm to 200 nm;

varying a lateral size of each of the 2D material (e.g., GO nanosheet) coatings to be at least 10 nm (e.g., 10 nm, 15 nm, 20 nm, 25 nm, 30 nm, 40 nm, 45 nm, 50 nm, 55 nm, 60 nm, 65 nm, 70 nm, 75 nm, 80 nm, 85 nm, 90 nm, 100 nm, 200 nm, 300 nm, 400 nm); 500 nm or less (e.g., 475 nm, 450 nm, 425 nm, 375 nm, 350 nm, 325 nm, 275 nm, 250 nm, 225 nm, 175 nm, 150 nm, 125 nm, 115 nm, 98 nm, 88 nm, 78 nm, 68 nm, 58 nm, 48 nm, 38 nm, 28 nm, 18 nm, 8 nm); or in a range of, from, including, and/or between 10 nm-500 nm (e.g., 7 nm-497 nm, 9 nm-397 nm, 13 nm-297 nm, 23 nm-295 nm, 33 nm-197 nm, 43 nm-195 nm, 53 nm-97 nm, 63 nm-95 nm, 73 nm-87 nm), or each of the GO nanosheet coatings may have a lateral size in a range of, from, including, and/or between 43 nm and 295 nm;

selecting or choosing a composition of the label, marker, biomarker, dye, or the like, such as but not limited to a Raman dye, where the label (e.g., Raman dye) comprises, consists essentially of, or consists of a cross-section value (e.g., Raman cross-section value) at the at least one excitation frequency at or greater than 1×10¹ Hz (e.g., 1×10² Hz, 1×10³ Hz, 1×10⁴ Hz, 1×10⁵ Hz, 1×10⁶ Hz, 1×10⁷ Hz, 1×10⁸ Hz, 1×10⁹ Hz, 1×10¹⁰ Hz, 1×10¹¹ Hz, 1×10¹² Hz, 1×10¹³ Hz, 1×10¹⁴ Hz, 2×10¹⁴ Hz, 3×10¹⁴ Hz, 4×10¹⁴ Hz, 5×10¹⁴ Hz, 1×10¹⁵ Hz, 1×10¹⁶ Hz, 1×10¹⁷ Hz, 1×10¹⁸ Hz, 1×10¹⁹ Hz, 1×10²⁰ Hz); 1×10²⁰ Hz or less (e.g., 1×10¹⁹ Hz, 1×10¹⁸ Hz, 1×10¹⁷, 1×10¹⁶ Hz, 1×10¹⁵ Hz, 5×10¹⁴ Hz, 4×10¹⁴ Hz, 3×10¹⁴ Hz, 2×10¹⁴ Hz, 1×10¹⁴ Hz, 1×10¹³ Hz, 1×10¹² Hz, 1×10¹¹ Hz, 1×10¹⁰ Hz, 1×10⁹ Hz, 1×10⁸ Hz, 1×10⁷ Hz, 1×10⁶ Hz, 1×10⁵ Hz, 1×10⁴ Hz, 1×10³ Hz, 1×10² Hz, 1×10¹ Hz); or in a range of, from, including, and/or between 1×10¹ Hz-1×10²⁰ Hz (e.g., 1×10² Hz-1×10¹⁹ Hz, 1×10³ Hz-1×10¹⁸ Hz, 1×10⁴ Hz-1×10¹⁷, 1×10⁵ Hz-1×10¹⁶ Hz, 1×10⁴ Hz-1×10¹⁵ Hz, 1×10⁵ Hz-5×10¹⁴ Hz, 1×10⁶ Hz-4×10⁷ Hz, 1×10⁷ Hz-3×10¹⁴ Hz, 1×10⁸ Hz-2×10¹⁴ Hz, 1×10⁹ Hz-1×10¹⁴ Hz, 1×10¹⁰ Hz-1×10¹³ Hz, 1×10¹¹ Hz, 1×10¹² Hz); or a Raman dye to have a Raman cross-section value of greater than 3×10¹⁴ Hz;

receiving, by a processor, from the detector, the at least one Raman shift in vibrational wavenumber (e.g., 2, 3, 4, 5, 6) of the at least one SERS spectra (e.g., 2, 3, 4, 5, 6), the at least one laser power intensity (e.g., second, third, fourth) of the at least one SERS spectra, or any combination thereof or data thereof (i.e., data about the at least one Raman shift in vibrational wavenumber of the at least one SERS spectra, the at least one laser power intensity of the at least one SERS spectra, or any combination thereof); Raman shift in vibrational wavenumber of SERS spectra to be detected by detector may be located at a range of, from, including, and/or between 100 cm⁻¹ to 10,000 cm⁻¹ (e.g., 100 cm⁻¹ to 1,000 cm⁻¹; 1,250 cm⁻¹ to 3,000 cm⁻¹; 500 cm⁻¹ to 3,250 cm⁻¹; 1,500 cm⁻¹ to 5,000 cm⁻¹; 3,500 cm⁻¹ to 10,000 cm⁻¹). The power density of a Raman laser may be located at a range of, from, including, and/or between 0.01 mW/cm² to 10 mW/cm² (e.g., 0.02 mW/cm², 0.1 mW/cm², 0.5 mW/cm², 1 mW/cm², 3 mW/cm², 9 mW/cm²). A Raman laser wavelength may be located at a range of, from, including, and/or between 250 nm to 3,000 nm (e.g., 270 nm, 325 nm, 405 nm, 488 nm, 532 nm, 575 nm, 633 nm, 780 nm, 808 nm, 980 nm, 1,200 nm, 2,000 nm); and

identifying, by the processor, the molecule or biochemical molecules in the sample based on the at least one Raman shift in vibrational wavenumber (e.g., 2, 3, 4, 5, 6) of the at least one SERS spectra, the at least one laser power intensity of the at least one SERS spectra, or any combination thereof.

Another embodiment of the disclosure may be directed to a method, where the detectable label, marker, biomarker, dye, or the like, or composition thereof, such as but not limited to a Raman dye may comprise, consist essentially of, or consisting of Cy5. The method of the disclosure may be directed to at least one molecule or a plurality of molecules (e.g., biochemical molecule; biomolecule; small molecule; drug; nucleic acids (e.g., DNA, DNA strand, DNA sequence, cDNA, RNA, RNA strand, DNA sequence, mRNA, miRNA); cell (e.g., stem cell, neural stem cell, differentiating cell, cell-derived vesicle); proteins; metabolites, pathogen, antigen, virus or viral particle: influenza virus (e.g., influenza A, zoonotic influenza, influenza B), respiratory syncytial virus, parainfluenza virus, adenovirus, rhinovirus, metapneumovirus, human metapneumovirus and endemic human coronaviruses (e.g., HKU1, OC43, NL63, 229E), enterovirus (e.g., EVD68), and coronavirus (e.g., MERS-CoV, SARS-CoV, SARS-CoV-2 or 2019-nCoV)). In some aspects, the method may utilize a substrate, where the substrate is glass or silicon, and substrate has deposited thereon, at least one or a plurality of plasmonic metal (e.g., Au, Ag, Pt, Al, Cu, Pd) protrusions or structures, including but not limited to nanostructures, having a shape or a structure selected from, for example, cones, obelisks, pyramids, inverted funnels, wedges, cylinders, rings, trees, branches, rods, wires, sheets, flowers, urchins, triangles, diamonds, stars, or the like.

Yet another embodiment may provide a method comprising, consisting essentially of, or consisting of, the steps of: disposing a sample onto a substrate or onto a plurality of plasmonic metal protrusions or structures; labeling at least one molecule or a plurality of molecules, in the sample with a detectable label; illuminating the at least one or plurality of plasmonic metal protrusions or structures with a light directed from an incident light source; detecting the labeled molecule; increasing a signal-to-noise ratio; receiving at least one Raman shift in vibrational wavenumber of the at least one SERS spectra, the at least one laser power intensity of the at least one SERS spectra, or any combination thereof; identifying the at least one molecule or a plurality of molecules; and further comprising the step(s) of: coating the plurality of plasmonic metal protrusions with 2D material (e.g., GO) nanosheets by applying electrostatic interactions using a chemical linker (e.g., cysteamine hydrochloride (C₂H₇NS.HCl)), where the plurality of plasmonic metal protrusions have a GO coating thickness may be 1 nm-2 nm; and/or forming the at least one or the plurality of plasmonic metal (e.g., Au, Ag, Pt, Al, Cu, Pd, Ge, Bi, Mg, Na, Li, Sn) protrusions or structures on the substrate (e.g., glass, silicon, plastics, fabrics/textiles, smart phone displays (e.g., aluminosilicate glass, alkali-aluminosilicate sheet glass, indium tin oxide)) using laser interference lithography and a physical vapor deposition (PVD) of the plasmonic metal (e.g., gold).

In some aspects, the forming step, where the forming at least one or the plurality of plasmonic metal protrusions or structures comprises plasmonic metal protrusions or structures that are cone-shaped or may have a shape selected from: obelisks, pyramids, inverted funnels, wedges, cylinders, rings, trees, branches, rods, wires, sheets, flowers, urchins, triangles, diamonds, stars, or the like. Other aspects of the embodiment may be directed to the cone-shaped plasmonic metal protrusion or structure having a width of at least 1 nm (e.g., 10 nm, 20 nm, 30 nm, 40 nm, 50 nm, 60 nm, 70 nm, 80 nm, 90 nm, 100 nm, 200 nm, 300 nm, 400 nm, 500 nm); 2,500 nm or less (e.g., 950 nm, 850 nm, 750 nm, 650 nm, 550 nm, 450 nm, 350 nm, 250 nm, 150 nm, 100 nm, 50 nm, 45 nm, 35 nm, 25 nm, 15 nm); or in a range of, from, including, and/or between 1 nm-2,500 nm (e.g., 10 nm-900 nm, 20 nm-800 nm, 30 nm-700 nm, 40 nm-600 nm, 50 nm-500 nm, 60 nm-400 nm, 70 nm-300 nm, 80 nm-200 nm, 90 nm-100 nm); or the cone-head plasmonic metal protrusion or structure may have a width of 250 nm; and/or a height of at least 10 nm (e.g., 11 nm, 20 nm, 30 nm, 40 nm, 50 nm, 60 nm, 70 nm, 80 nm, 90 nm, 100 nm, 200 nm, 300 nm, 400 nm, 500 nm); 20,000 nm or less (e.g., 19,950 nm, 10,000 nm, 8,500 nm, 1,000 nm, 750 nm, 650 nm, 550 nm, 450 nm, 350 nm, 250 nm, 150 nm, 100 nm, 50 nm, 45 nm, 35 nm, 25 nm, 15 nm); or in a range of, from, including, and/or between 10 nm-20,000 nm (e.g., 15 nm-19,500 nm, 25 nm-9,000 nm, 35 nm-8,000 nm, 45 nm-7,000 nm, 55 nm-6,000 nm, 65 nm-5,000 nm, 75 nm-4,000 nm, 85 nm-3,000 nm, 95 nm-2,000 nm, 105 nm-1,000 nm); or the plasmonic metal protrusion or structure may extend from the substrate or the surface of the substrate to a height of 100 nm.

Another aspect of the embodiments of the disclosure may be directed to a method comprising, consisting essentially of, or consisting of, the steps of: disposing a sample onto a substrate or onto a plurality of plasmonic metal protrusions or structures; labeling at least one molecule or a plurality of molecules, in the sample with a detectable label; illuminating the at least one or plurality of plasmonic metal protrusions or structures with a light directed from an incident light source; detecting the labeled molecule; increasing a signal-to-noise ratio; receiving at least one Raman shift in vibrational wavenumber of the at least one SERS spectra, the at least one laser power intensity of the at least one SERS spectra, or any combination thereof; identifying the at least one molecule or a plurality of molecules; and where the at least one molecule or plurality of molecules (e.g., biochemical molecule; biomolecule; small molecule; drug; nucleic acids (e.g., DNA, DNA strand, DNA sequence, cDNA, RNA, RNA strand, DNA sequence, mRNA, miRNA); cell (e.g., stem cell, neural stem cell, differentiating cell, cell-derived vesicle); proteins; metabolites, pathogen, antigen, virus or viral particle: influenza virus (e.g., influenza A, zoonotic influenza, influenza B), respiratory syncytial virus, parainfluenza virus, adenovirus, rhinovirus, metapneumovirus, human metapneumovirus and endemic human coronaviruses (e.g., HKU1, OC43, NL63, 229E), enterovirus (e.g., EVD68), and coronavirus (e.g., MERS-CoV, SARS-CoV, SARS-CoV-2 or 2019-nCoV)) or biochemical molecule comprising, consisting essentially of, or consisting of neural stem cells, further comprising the step of monitoring, by a processor, changes in a SERS signal spectrum or SERS spectra for characterizing neural stem cell differentiation.

### Applications

The system and methods of the disclosure as described here may be applied to or used to identify at least one molecule or a plurality of molecules (e.g., biochemical molecule; biomolecule; small molecule; drug; nucleic acids (e.g., DNA, DNA strand, DNA sequence, cDNA, RNA, RNA strand, DNA sequence, mRNA, miRNA); cell (e.g., stem cell, neural stem cell, differentiating cell, cell-derived vesicle); proteins; metabolites, pathogen, antigen, virus or viral particle: influenza virus (e.g., influenza A, zoonotic influenza, influenza B), respiratory syncytial virus, parainfluenza virus, adenovirus, rhinovirus, metapneumovirus, human metapneumovirus and endemic human coronaviruses (e.g., HKU1, OC43, NL63, 229E), enterovirus (e.g., EVD68), and coronavirus (e.g., MERS-CoV, SARS-CoV, SARS-CoV-2 or 2019-nCoV)), chemical bonds, and intramolecular bonds, or a fingerprint region based on vibrational frequencies that are specific to the bonds of a molecule.

One embodiment may provide the graphene-plasmonic hybrid nanoarray as a SERS substrate for a wide range of applications, including a simple, rapid, and accurate sensing platform for screening a wide variety of biological/chemical molecules. This may include, more specifically, biomolecular interactions within cells that play a vital role in biological processes that can be helpful markers for disease diagnostics and drug screening. Gene and protein sensing for disease detection and basic stem cell research.

The available techniques using surface-enhanced Raman scattering (SERS) lack uniform distribution of the plasmonic metal (e.g., noble metal) structures (e.g., nanostructures) in the large-scale, which leads to irreproducible electromagnetic mechanism (EM)-based enhancement. Mismatched surface energy levels of the nanostructure and adsorbates (i.e., molecules of interest) can also significantly reduce the CM-based enhancement by limiting the charge transfer, thus hindering the practical application of SERS-based sensing. In comparison, the disclosed system and methods of use thereof as described here are advantageous over the available techniques because the methods of the disclosure produce homogeneous SERS substrates which result in a homogeneous SERS signal distribution with a low standard deviation (e.g., below 0.1).

Some embodiments of the disclosure provide a rapid, sensitive, and non-destructive spectroscopic method for trace analyses, which exhibits use in a wide variety of applications in diagnosis, biomedicine, catalysis monitoring, environmental analysis, food safety, and bio/chemical sensing.

In one embodiment, the system and methods of the disclosure may be used to identify a plethora of molecules including but not limited to: nucleic acids, DNA, viral DNA, ssDNA, modifications in ssDNA, radical S-Adenosyl methionine domain containing 2 (RSAD2) gene, bacterial DNA, RNA, microRNA, miR-21, amino acids, proteins, carcinoembryonic antigen (CEA), alpha-fetoprotein (AFP), disease biomarkers (e.g., cancer biomarkers: MUC4), prostate-specific antigen (PSA), neurotransmitters, peptides, drugs, active pharmaceutical ingredients (APIs) and their polymorphic forms, pathogenic bacteria, cells (e.g., stem cells, neural stem cells, differentiating cells, cell-derived vesicles), virus or viral particles. Non-limiting examples of samples comprising at least one or a plurality of molecules include, blood, saliva, urine, semen, or samples with at least one molecule or a plurality of molecules isolated or extracted from: hair, tissue, bone, teeth, and the like of a subject (e.g., mammal) or soil, textile, glass, and the like.

### EXAMPLES

The following examples illustrate specific aspects of the instant description. The examples should not be construed as limiting, as the example merely provides specific understanding and practice of the embodiments and its various aspects.

### Example 1: Generation of Au Nanoarray Using Laser Interference Lithography

To generate the homogeneous polymer nanoarray on a large scale as a template for the SERS substrate, laser interference lithography (LIL) was used. Briefly, one by one-centimeter square (1×1 cm²) glass substrates were sequentially washed by 1% Triton-X aqueous solution, ethanol solution, and distilled deionized water (DIW) for one hour under bath sonication and dried under N₂ gas. To increase the adhesion between the substrate and photoresist, a layer of hexamethyldisilazane (HMDS) ((CH₃)₃ SiNHSi (CH₃)₃) was functionalized through vapor phase deposition. Then a UV-cross-linkable positive photoresist (PR) solution was spin-coated on the HMDS pre-coated glass substrate for 40 seconds at 4,000 rpm. PR coated glass substrate was soft baked on a hot plate at 120° C. for the 60 seconds and then exposed to UV (λ=325 nm, 0.81 mW) using Lloyd's mirror interferometer. Lloyd's mirror interferometer was utilized to generate periodic intensity profiles through constructive/destructive interference which occurred between the light coming from the light source (He—Cd laser, KIMMON KOHA Laser Systems, Japan) and the light reflected from the mirror. The angle of sample holder incorporating Lloyd's mirror was adjusted to generate PR nanopatterns with different sizes according to the equation given by the formula:

Λ=λ/2 Sin θ (3)

where Λ, λ, and θ are the size of the pitch (nm), a wavelength of the UV laser (325 nm), and the incident angle (°), respectively. The incident angles were adjusted at 26.0° to generate the pitch sizes of 500 nm. For the generation of PR nanohole patterns, the substrate was double-exposed to UV with a degree of sample rotation (0°, 90°) for 25 seconds each followed by 30 seconds developing and washing with distilled deionized water (D1W). A hologram from the photoresist nanoarray should appear at this stage.

To generate the gold (Au) nanoarray, the polymer nanoarray was utilized as a template and gold were deposited onto the polymer substrate via physical vapor deposition. To control the layer thickness of the gold, different deposition times of 120 s, 240 s, 480 s, 960 s, and 1200 s were used to deposit 10 nm, 20 nm, 40 nm, 80 nm, and 100 nm of gold, respectively. The developed substrates were characterized by field emission scanning electron microscope (FE-SEM, Zeiss) and atomic force microscope (AFM, Park systems, NX10).

### Example 2: Synthesis of Graphene Oxide Nanosheets

Graphene oxide nanosheet was synthesized using a protocol with minor modifications. Graphite oxide was first generated from graphite. Briefly, 0.5 gram 100 mesh-sized graphite was added into 6 ml 98% H₂SO₄, 1.3 g K₂S₂O₈ and 1.3 g P₂O₅ at 80° C. for 8 hours. Then, 250 mL of ultrapure water was slowly added and the mixed solution was vigorously stirred for 12 hours to get the pre-oxidized graphite. Afterward, the black colored mixture was centrifuged down and dried at room temperature overnight. To synthesize the graphite oxide, the dried and pre-oxidized graphite was slowly mixed with 10 ml concentrated (98%) H₂SO₄. Twenty minutes after the stirring, 8 g KMnO₄ was added into the mixture and stirred over one hour. All reactions were performed under ice bath conditions to keep the temperature of the reaction below 15° C. After one hour stirring, the temperature of the reaction was increased to 35° C. using a hotplate and continuously stirred for 5 hours. De-ionized water (150 ml) was added to the mixture drop by drop (where the temperature cannot exceed 50° C.) and vigorously stirred for 6 hours. Lastly, 500 ml of de-ionized water was slowly added to the reaction and 20 ml 30% H₂O₂ aqueous solution was injected to quench the reaction. A shining yellow colored solution should appear at this stage, indicating the successful formation of graphite oxide. To purify the graphite oxide, the solution was centrifuged at 10,000 rpm for 5 minutes and then washed with 12% HCl solution three times, followed by five times washing with de-ionized water to remove the HCl residues. To obtain the graphene oxide, 10 hours of ultrasonication was performed to remove the aggregated graphite oxide, the final solution was centrifuged at 10,000 rpm for 30 minutes and the supernatant was collected. The concentration of graphene oxide (GO) by drying 1.0 ml of the solution and weigh the solid after evaporation of water. The GO was then characterized by transmission electron microscope (TEM), dynamic light scattering (DLS) analysis (Nanosizer, Malvern Instruments).

### Example 3: Finite Difference Time Domain (FDTD) Simulation

The electromagnetic enhancement of SERS was performed by FDTD simulation using parameters based on SEM and AFM characterizations. Briefly, a 3D model for hollow nanocones with the composition of gold was built. Afterwards, a four by four array was built on top of a substrate with the composition of silicon. The laser wavelength used in the SERS measurements was matched with the light source (plane wave) by setting the wavelengths at 514 nm, 633 nm, and 780 nm and at vertical directions to the substrate from top. In the FDTD simulation, 4 nm mesh size was defined as the mesh size and perfectly matched layer (PML) with 12-layer numbers were used as boundary conditions. Both vertical and parallel monitors were incorporated to obtain results and monitoring wavelengths were kept consistent with the light sources.

### Example 4: Generation of Combinatorial Graphene-Au Hybrid Nanoarray

To fabricate the combinatorial graphene-Au hybrid nanoarray, an electrostatic assembly strategy was used. Briefly, the surface of Au nanoarray was functionalized with cysteamine aqueous solution (concentration of 1.0 mg/ml) through the thiol-gold interactions overnight. After extensive washing in water to remove the residual cysteamine molecules, nano graphene oxide solution (concentration of 3.0 mg/ml) was added onto the Au nanoarray and incubated for 24 hours. Then the excessive amount of GO was washed away using de-ionized water. To perform the reduction of GO on the Au nanoarray, ascorbic acid and hydrazine-based reduction were used, respectively. For the ascorbic acid-based reduction, GO functionalized Au nanoarray was incubated with an ascorbic acid aqueous solution at a concentration of 1.0 mg/ml for a different period of 12 hours, 24 hours, and 48 hours to modulate the reduction level and energy levels of reduced graphene oxide (rGO). To achieve the highest reduction level, hydrazine vapor-based reduction was used. In a sealed glass dish, the GO functionalized Au nanoarray was placed in proximity with a drop of hydrazine solution. Afterward, the dish was heated to 80° C. for 24 hours. It is known that excess time of reduction of GO by hydrazine can generate a high reduction level similar to the pristine graphene with the working function of −4.5 eV. The combinatorial graphene-Au hybrid nanoarray with different reduction levels was characterized using Raman spectrum using a laser at 633 nm with background subtracted using a polynomial function in the Labspec software. Intensity of the 633 nm laser was kept at 0.087 mW unless specifically mentioned (e.g., intensity is different in the Raman mapping experiments).

In the steps previously mentioned, to ensure the uniformity of GO coating on the Au nanoarray in three aspects. First, both steps were performed in highly concentrated solutions and the assembly dynamics and kinetics were purely driven by the nature of covalent bonding or electrostatic interactions which are uniform across the surfaces of the substrate. Since the concentration of reactants (cysteamine and GO) was high, the concentration was assumed constant across the whole assembly process on the interfaces of whole substrate. In contrast, physical deposition methods such as dip-coating or drop-casting typically cause interferences from evaporation which can lead to inhomogeneity of GO coating. Second, in each experiment, the reactants were extensively washed and aggregates were removed that might cause inhomogeneous coating. Third, nano-sized GO (<200 nm) that is dramatically smaller than the laser diameter (>2 μm) was synthesized. Therefore, the small variance in GO structure and coating might be negligible in SERS measurements.

To study the thickness-dependent SERS effects on the Au nanoarray based on the electromagnetic mechanism, GO was directly used as a Raman dye. GO was coated onto the Au nanoarray as well as a control substrate of planar gold using the identical procedure of electrostatic assembly described previously. Afterward, the GO-coated nanoarrays with different thicknesses of gold were analyzed by Raman using a 633 nm laser. The intensities of the graphite peak at 1600 cm⁻¹ were averaged from three individual measurements and then converted into a bar graph in FIG. 2C.

To characterize the layer thickness of GO after being assembled to the Au nanoarray, the graphene-Au hybrid nanoarray was scratched followed by extensive tip sonication in water solution. Then the solution was drop-cast to the transmission electron microscopy (TEM) copper grids (EMS). A Philips CM12 electron microscope with AMT-XR11 digital camera was used for the characterization of GO assembled on the nanoarray. For the Raman measurements in FIG. 3B, a 633 nm laser with laser power density of 0.087 mW with an accumulation time of 10 seconds was used for the spectrum collection (FIG. 3B) and accumulation time of 1 second for the mapping. The differences in Raman signal intensity (e.g., 10 counts; 100 counts; 3000 counts; 10,000 counts; 70,000 counts; 200,000 counts; 350,000 counts) of the graphene peaks originated from the two different measurement techniques (Raman spectrum collection vs. Raman mapping) and accumulation time instead of the substrate variations.

### Example 5: Density Functional Theory (DFT) Simulation

To calculate the energy levels of Cy5 Raman dye, the energy and frequency of Cy5 molecules were calculated using the B3LYP method with a 3-21G basis set in the Gaussian 09 software. The Cy5 dye has one positive charge and is in a triplet spin state. The total energy of Cy5 was calculated to be −1494.41172095 arbitrary units (a.u.) with root mean square (RMS) gradient norm of 0.00000198 a.u. The detailed atomic coordination of Cy5 was provided. Similarly, to show the effect of reduction on a graphene-like structure, DFT simulation was also performed on a structure with nine fused benzene rings as well as its derivatives with four hydroxyl groups (HOMO: −4.21613 eV; LUMO: −0.0774 eV), four carboxylic groups (HOMO: −6.72938 eV; LUMO: −0.73035 eV), four epoxy groups (HOMO: −5.08363 eV; LUMO: −3.14754 eV) or combination form (HOMO: −7.74708 eV; LUMO: −0.08299 eV). These functional groups are commonly seen in graphene oxide and the coordination and calculation summaries can be found in the supporting information. The highest occupied molecular orbital (HOMO) and lowest unoccupied molecular orbital (LUMO) of each compound listed were obtained from the molecular orbitals of the optimized form.

TABLE 1
Coordinates for optimized Cy5 molecule

| Center | Atomic | Atomic | Coordinates (Angstroms) | | |
|---|---|---|---|---|---|
| No. | No. | Type | X | Y | Z |
| 1 | 6 | 0 | −4.573036 | −2.429400 | −0.368585 |
| 2 | 6 | 0 | −3.880795 | −3.509952 | 0.205765 |
| 3 | 6 | 0 | −4.571384 | −4.638056 | 0.615097 |
| 4 | 6 | 0 | −5.963811 | −4.677427 | 0.447377 |
| 5 | 6 | 0 | −6.644522 | −3.594139 | −0.123302 |
| 6 | 6 | 0 | −5.958094 | −2.451905 | −0.540050 |
| 7 | 6 | 0 | −2.356117 | −1.781791 | −0.355211 |
| 8 | 1 | 0 | −4.051616 | −5.478908 | 1.058836 |
| 9 | 1 | 0 | −6.518407 | −5.552223 | 0.762730 |
| 10 | 1 | 0 | −7.719737 | −3.639964 | −0.243527 |
| 11 | 1 | 0 | −6.494008 | −1.619518 | −0.976412 |
| 12 | 6 | 0 | −1.262039 | −0.954513 | −0.560738 |
| 13 | 1 | 0 | −1.458040 | 0.016256 | −1.008936 |
| 14 | 6 | 0 | 0.085652 | −1.221342 | −0.251250 |
| 15 | 1 | 0 | 0.360793 | −2.166712 | 0.199906 |
| 16 | 6 | 0 | 1.101183 | −0.293654 | −0.505400 |
| 17 | 1 | 0 | 0.811005 | 0.654157 | −0.960074 |
| 18 | 6 | 0 | 2.460281 | −0.479944 | −0.222514 |
| 19 | 1 | 0 | 2.762162 | −1.416561 | 0.229844 |
| 20 | 6 | 0 | 3.422708 | 0.505467 | −0.507788 |
| 21 | 1 | 0 | 3.045573 | 1.415958 | −0.967613 |
| 22 | 6 | 0 | 4.788021 | 0.448406 | −0.269141 |
| 23 | 6 | 0 | 7.018202 | −0.084935 | 0.329437 |
| 24 | 6 | 0 | 6.974377 | 1.193947 | −0.254874 |
| 25 | 6 | 0 | 8.224140 | −0.610039 | 0.761908 |
| 26 | 6 | 0 | 8.125863 | 1.964884 | −0.416922 |
| 27 | 6 | 0 | 9.390759 | 0.153340 | 0.605776 |
| 28 | 1 | 0 | 8.272774 | −1.593471 | 1.214476 |
| 29 | 6 | 0 | 9.336527 | 1.425378 | 0.022118 |
| 30 | 1 | 0 | 8.099342 | 2.949335 | −0.864711 |
| 31 | 1 | 0 | 10.341459 | −0.243228 | 0.939106 |
| 32 | 1 | 0 | 10.245643 | 2.002624 | −0.091482 |
| 33 | 6 | 0 | 5.614215 | −0.680340 | 0.371270 |
| 34 | 6 | 0 | 5.187685 | −0.933590 | 1.843400 |
| 35 | 1 | 0 | 5.249961 | −0.007645 | 2.421342 |
| 36 | 1 | 0 | 4.164768 | −1.314973 | 1.895758 |
| 37 | 1 | 0 | 5.858773 | −1.673112 | 2.292131 |
| 38 | 6 | 0 | 5.561491 | −1.978458 | −0.480559 |
| 39 | 1 | 0 | 4.549544 | −2.390608 | −0.506026 |
| 40 | 1 | 0 | 5.885633 | −1.774453 | −1.504618 |
| 41 | 1 | 0 | 6.231134 | −2.725929 | −0.043000 |
| 42 | 7 | 0 | 5.642987 | 1.490069 | −0.602560 |
| 43 | 6 | 0 | 5.213190 | 2.746885 | −1.237711 |
| 44 | 1 | 0 | 4.747693 | 2.539887 | −2.206195 |
| 45 | 1 | 0 | 4.501788 | 3.271786 | −0.592673 |
| 46 | 1 | 0 | 6.083642 | 3.381965 | −1.393069 |
| 47 | 7 | 0 | −3.652258 | −1.419515 | −0.705425 |
| 48 | 6 | 0 | −4.060296 | −0.120988 | −1.285420 |
| 49 | 1 | 0 | −3.288036 | 0.201878 | −1.988728 |
| 50 | 1 | 0 | −4.976498 | −0.291900 | −1.856220 |
| 51 | 6 | 0 | −4.297500 | 0.951742 | −0.198018 |
| 52 | 1 | 0 | −5.052369 | 0.584858 | 0.507070 |
| 53 | 1 | 0 | −3.368529 | 1.107711 | 0.363015 |
| 54 | 6 | 0 | −4.767310 | 2.286099 | −0.817592 |
| 55 | 1 | 0 | −4.027012 | 2.633383 | −1.551988 |
| 56 | 1 | 0 | −5.711206 | 2.126679 | −1.356614 |
| 57 | 6 | 0 | −4.965018 | 3.370616 | 0.260948 |
| 58 | 1 | 0 | −5.684982 | 3.020406 | 1.010113 |
| 59 | 1 | 0 | −4.023082 | 3.549585 | 0.791357 |
| 60 | 6 | 0 | −5.461396 | 4.700574 | −0.326796 |
| 61 | 1 | 0 | −6.456942 | 4.601620 | −0.773675 |
| 62 | 1 | 0 | −4.791681 | 5.057314 | −1.120524 |
| 63 | 6 | 0 | −5.522867 | 5.767111 | 0.745932 |
| 64 | 8 | 0 | −5.124599 | 5.658410 | 1.899621 |
| 65 | 8 | 0 | −6.091287 | 6.916977 | 0.244267 |
| 66 | 1 | 0 | −6.114077 | 7.605850 | 0.965725 |
| 67 | 6 | 0 | −2.390962 | −3.190667 | 0.265718 |
| 68 | 6 | 0 | −1.913734 | −3.172654 | 1.744209 |
| 69 | 1 | 0 | −0.859898 | −2.891872 | 1.814478 |
| 70 | 1 | 0 | −2.507556 | −2.462560 | 2.326135 |
| 71 | 1 | 0 | −2.037668 | −4.170715 | 2.176890 |
| 72 | 6 | 0 | −1.593353 | −4.211506 | −0.591583 |
| 73 | 1 | 0 | −1.961660 | −4.212625 | −1.621022 |
| 74 | 1 | 0 | −0.527056 | −3.971670 | −0.598170 |
| 75 | 1 | 0 | −1.722009 | −5.214467 | −0.171844 |

The energy levels of methylene blue were calculated using identical procedures.

TABLE 2
Coordinates for optimized methylene blue molecule

| Center | Atomic | Atomic | Coordinates (Angstroms) | | |
|---|---|---|---|---|---|
| No. | No. | Type | X | Y | Z |
| 1 | 16 | 0 | 0.000000 | 1.381573 | 0.000000 |
| 2 | 7 | 0 | −5.185054 | 0.662341 | 0.000000 |
| 3 | 7 | 0 | 5.295337 | 0.457068 | 0.000000 |
| 4 | 7 | 0 | −0.094576 | −1.777137 | 0.000000 |
| 5 | 6 | 0 | −1.438728 | 0.317208 | 0.000000 |
| 6 | 6 | 0 | 1.367348 | 0.225648 | 0.000000 |
| 7 | 6 | 0 | −1.243250 | −1.158868 | 0.000000 |
| 8 | 6 | 0 | 1.175411 | −1.129748 | 0.000000 |
| 9 | 6 | 0 | −3.920058 | 0.037841 | 0.000000 |
| 10 | 6 | 0 | 3.831839 | −0.043740 | 0.000000 |
| 11 | 6 | 0 | −2.681729 | 0.875499 | 0.000000 |
| 12 | 6 | 0 | 2.723515 | 0.786321 | 0.000000 |
| 13 | 6 | 0 | −2.461927 | −1.984096 | 0.000000 |
| 14 | 6 | 0 | 2.336509 | −2.009675 | 0.000000 |
| 15 | 6 | 0 | −3.701035 | −1.434007 | 0.000000 |
| 16 | 6 | 0 | 3.599146 | −1.505981 | 0.000000 |
| 17 | 6 | 0 | −6.485775 | −0.219734 | 0.000000 |
| 18 | 6 | 0 | −5.324182 | 2.241603 | 0.000000 |
| 19 | 6 | 0 | 6.468641 | −0.635345 | 0.000000 |
| 20 | 6 | 0 | 5.615184 | 1.957304 | 0.000000 |
| 21 | 1 | 0 | −2.757646 | 1.954488 | 0.000000 |
| 22 | 1 | 0 | 2.800492 | 1.862584 | 0.000000 |
| 23 | 1 | 0 | −2.339296 | −3.056189 | 0.000000 |
| 24 | 1 | 0 | 2.169959 | −3.076260 | 0.000000 |
| 25 | 1 | 0 | −4.542002 | −2.107798 | 0.000000 |
| 26 | 1 | 0 | 4.428985 | −2.193126 | 0.000000 |
| 27 | 1 | 0 | −5.962425 | −1.138089 | 0.000000 |
| 28 | 1 | 0 | −7.576359 | −0.183186 | 0.000000 |
| 29 | 1 | 0 | −6.485848 | 0.885796 | 0.000000 |
| 30 | 1 | 0 | −6.411710 | 2.299975 | 0.000000 |
| 31 | 1 | 0 | −4.740435 | 3.155677 | 0.000000 |
| 32 | 1 | 0 | −4.357670 | 1.670460 | 0.000000 |
| 33 | 1 | 0 | 6.688545 | 0.458946 | 0.000000 |
| 34 | 1 | 0 | 7.542536 | −0.868510 | 0.000000 |
| 35 | 1 | 0 | 5.748335 | −1.424190 | 0.000000 |
| 36 | 1 | 0 | 4.566719 | 1.584739 | 0.000000 |
| 37 | 1 | 0 | 5.234711 | 2.979313 | 0.000000 |
| 38 | 1 | 0 | 6.717709 | 1.772928 | 0.000000 |

### Example 6: DNA Detection by Combinatorial Graphene-Au Hybrid SERS Nanoarray

To detect target DNA, probe DNA with complementary sequences conjugated with Cy5 was designed (Integrated DNA Technologies, USA). To immobilize the Cy5-labeled probe DNA onto the combinatorial graphene-Au hybrid nanoarray, different concentrations of probe DNA (1 nM, 10 nM, 100 nM, 1 μM, 10 μM and 100 μM) were used to incubate with the nanoarray followed by drying in the vacuum condition. All SERS experiments were performed right after the absorption and nuclease free buffer was prepared from nuclease-free water (Qiagen) to avoid the potential oxidation or degradation of the probe DNA. To detect the target DNA, the combinatorial graphene-Au hybrid SERS nanoarray immobilized with probe DNA (100 μM) was incubated with target DNA sequence with different concentrations (range from 100 fM to 10 μM) or with a single-mismatch DNA sequence at a concentration of 10 nM in the nuclease-free buffer (PBS) at room temperature. The DNA detections were all performed under a 633 nm laser with laser power density of 0.087 mW with accumulation time of 10 seconds except for the single-base mismatch study where Raman mapping was performed at the laser intensity of 0.31 mW with accumulation time of 1 second to obtain sufficient signals (FIG. 12). Then the Raman spectrum at three randomly chosen spots on each substrate was collected with background subtraction using the Labspec software with identical protocol aforementioned. For the SERS signal stability test, substrates incubated with 1 μM Cy5-DNA were measured with Raman intensity at 6 randomly selected spots before and after the hybridization time (one hour) and then the spectrum were summarized in FIG. 9. To quantify the intensity change of the Raman spectrum, the identical Raman transition band at 1120 cm⁻¹ of the Cy5 dye was used as the standard peak followed by background subtraction using Labspec software.

To perform gene analysis for the neuronal differentiation of human neural stem cells, cell lysates from the stem cells or the differentiated cells were collected through Trizol. The mRNA in the cell lysates were converted into cDNA. cDNA was detected using Raman on the combinatorial graphene-Au hybrid nanoarray following identical protocol mentioned above. Three randomly selected spots were used for the Raman analysis and obtain the statistical information.

### Example 7: Cell Culture and Differentiation

Human neural stem cells (hNSCs) were maintained in a mixture of neural basal medium (Gibco) and DMEM/F12 (Gibco) (50:50 ratio) supplemented with 0.5% N2 (Gibco), 0.5% B27, and 20 ng/mL FGF basic (Fibroblast growth factor-basic, PeproTech) respectively. All cells were maintained at 37° C. in a humidified incubator with 5% CO₂. To differentiate cells, hNSCs were seeded on matrigel (Life Technologies) pre-coated plates (ca. 80,000 cells/well for 24-well plate) 24 hr before experimentation. After one day of cultivation to promote cell attachment and spreading, the fresh hNSC media without FGF basic (differentiation media) was treated to stop proliferation and induce neuronal differentiation. The medium was changed with fresh differentiation media every 3-4 days during the differentiation. For consistency, all experiments were carried out on cells between 3 passage differences.

### Example 8: Immunocytochemistry

To study the extent of neuronal differentiation, cells are washed with DPBS (pH 7.4) and fixed with 4% formaldehyde solution for 10 min at room temperature (RT), followed by three times of washing with DPBS. Then, cells were permeabilized with 0.1% Triton X-100 in PBS for 10 minutes and non-specific binding is blocked with 5% normal goat serum (NGS) (eLife Technologies) in PBS for 1 hour at room temperature. The primary rabbit antibody against Nestin (1:200 dilution, Invitrogen), the primary mouse antibody against TuJ1 (1:200 dilution, Biolegend) and primary rabbit antibody against GFAP (1:200 dilution, Invitrogen) were used.

Following the manufacturer's protocol, the fixed samples were incubated overnight at 4° C. in a solution of this antibody in PBS containing 1% BSA and 0.3% Triton X-100. After washing three times with PBS, the samples were incubated for 1 hr at room temperature in a solution of anti-mouse secondary antibody labeled with Alexa Flour 594 (1:200, Life Technologies), anti-rabbit secondary antibody labeled Alexa Flour 488 (1:200, Life Technologies), and Hoechst (3 μg/mL, Life Technologies) to stain the nucleus in PBS containing 1% NGS and 0.3% Triton X-100. After washing three times, all the samples were imaged using the Nikon T2500 inverted fluorescence microscope.

### Example 9: Gene Expression Analysis

Gene expression levels were analyzed by quantitative reverse transcription PCR (RT-qPCR) from total RNA extracted from cells by a TRIzol reagent (Invitrogen, MA). The total RNA (1 μg) was reverse transcribed to cDNA using the SuperScript III First-Strand Synthesis System (Invitrogen, MA) following the manufacturer's protocol. Subsequently, quantitative PCR was performed on a StepOnePlus Real-time PCR System (Applied Biosystems, MA) using an SYBR Green PCR Master Mix (Applied Biosystems, MA) with the gene-specific primers, listed in FIG. 13. Standard cycling conditions were used for all PCR reactions with a melting temperature of 60° C. All the measurements were run in triplicate. The gene expression levels were reported relative to the endogenous control gene, GAPDH.

### Example 10: Gene Expression Analysis

The melting temperature (Tm) for the matched and mismatched DNA hybridization was calculated using NUPACK software at the designated DNA concentration (10 nM), which provides information on the thermodynamics at hybridization. The simulated results summarized a lower Tm for the double strand with a single base pair mismatch (G to C, 68.5° C.) compared to the one with a perfect complementary match (77.5° C.) in agreement with literature reports on the thermodynamics of DNA hybridization. Also, at the experimental temperature (25° C.), the unpaired fraction from the mismatched condition (0.15374) was significantly higher compared to the matched condition (0.04771), which supported the Raman measurement result that distinguishes the single base pair mismatch.

### Specific Embodiments

Non-limiting specific embodiments are described here each of which is considered to be within the present disclosure.

As various changes can be made in the above-described subject matter without departing from the scope and spirit of the present disclosure, it is intended that all subject matter contained in the above description, or defined in the appended claims, be interpreted as descriptive and illustrative of the present disclosure. Many modifications and variations of the present disclosure are possible in light of the above teachings. Accordingly, the present description is intended to embrace all such alternatives, modifications and variances which fall within the scope of the appended claims.

All documents cited or referenced herein and all documents cited or referenced in the herein cited documents, together with any manufacturer's instructions, descriptions, product specifications, and product sheets for any products mentioned herein or in any document incorporated by reference herein, are hereby incorporated by reference, and may be employed in the practice of the disclosure.

1. A system, comprising:
a nanoarray, comprising:
a substrate, wherein the substrate is glass; and
a plurality of plasmonic metal protrusions extending from the substrate;
wherein the plurality of plasmonic metal protrusions is of a plasmonic metal that is gold (Au);
wherein the plasmonic metal in each of the plurality of plasmonic metal protrusions has a thickness in a range between 20 nm-200 nm;
wherein the plurality of plasmonic metal protrusions has a respective plurality of graphene oxide (GO) nanosheet coatings layered thereupon;
wherein each of the GO nanosheet coatings has a lateral size in a range between 43 nm and 295 nm.

2. The system according to claim 1, wherein a sample comprising biochemical molecules are located on the plurality of plasmonic metal protrusions.

3. The system according to claim 2, wherein the biochemical molecules in the sample are selected from the group consisting of: cells, cell-derived vesicles, RNA sequences, DNA sequences, pathogens, antigens, viruses, and viral particles.

4. The system according to claim 2, wherein the biochemical molecules in the sample comprise:
i) a Raman dye label; and
ii) a coupling to at least a portion of the plurality of plasmonic metal protrusions.

5. The system according to claim 2, wherein the biochemical molecules emit at least one surface enhanced Raman scattering (SERS) light in response to a light directed onto the plurality of plasmonic metal protrusions from an incident light source.

6. A system, comprising:
a nanoarray, comprising:
a substrate;
a plurality of plasmonic metal protrusions extending from the substrate;
wherein the plurality of plasmonic metal protrusions has a respective plurality of graphene oxide (GO) nanosheet coatings layered thereupon;
a sample comprising biochemical molecules located on the plurality of plasmonic metal protrusions;
wherein the biochemical molecules in the sample are:
i) labeled with a Raman dye; and
ii) coupled to at least a portion of the plurality of plasmonic metal protrusions;
an incident light source configured to direct a light, having at least one excitation frequency, onto the plurality of plasmonic metal protrusions;
wherein the biochemical molecules emit at least one surface enhanced Raman scattering (SERS) light in response to the light being directed onto the plurality of plasmonic metal protrusions from the incident light source;
a detector configured to detect at least one laser power intensity and at least one Raman shift in vibrational wavenumber of the at least one Raman dye in a SERS spectra;
wherein a signal-to-noise ratio (SNR) of the at least one SERS spectra is above a SNR predefined threshold when:
a thickness of a plasmonic metal in each of the plurality of plasmonic metal protrusions is in a range between 20 nm to 200 nm;
a lateral size of each of the GO nanosheet coatings is in a range between 5 nm and 20,000 nm; and
a composition of the Raman dye is chosen to have a Raman cross-section value at the at least one excitation frequency greater than a 3×10¹⁴ Hz; and
a processor configured to:
i) receive, from the detector, data about the at least one Raman shift in vibrational wavenumber of the at least one SERS spectra, the at least one laser power intensity of the at least one SERS spectra, or any combination thereof, and
ii) identify the biochemical molecules in the sample based on the at least one Raman shift in vibrational wavenumber of the at least one SERS spectra, the at least one laser power intensity of the at least one SERS spectra, or any combination thereof.

7. The system according to claim, wherein the composition of the Raman dye comprises Cy5.

8. The system according to claim, wherein the biochemical molecules in the sample are selected from the group consisting of: cells, cell-derived vesicles, RNA sequences, DNA sequences, pathogens, antigens, viruses, and viral particles.

9. The system according to claim, wherein the SNR predefined threshold is 34.

10. The system according to claim, wherein each plasmonic metal protrusion extending from the substrate is cone-shaped.

11. The system according to claim 10, wherein each plasmonic metal protrusion that is cone-shaped has a width of 250 nm and a height of 100 nm.

12. The system according to claim, wherein the substrate is glass.

13. The system according to claim, wherein the plasmonic metal is gold.

14. The system according to claim 13, wherein the plurality of GO nanosheet coatings of the plurality of plasmonic metal protrusions have a thickness in a range between 1 nm and 2 nm.

15. The system according to claim, wherein the biochemical molecules comprise neural stem cells, and wherein the processor is further configured to monitor changes in the SERS spectra for characterizing neural stem cell differentiation.

16. A method, comprising:
disposing a sample onto a plurality of plasmonic metal protrusions extending from a substrate;
wherein the plurality of plasmonic metal protrusions has a respective plurality of graphene oxide (GO) nanosheet coatings;
wherein the sample comprises biochemical molecules that are coupled to at least a portion of the plurality of plasmonic metal protrusions;
labeling the biochemical molecules in the sample with a Raman dye;
illuminating the plurality of plasmonic metal protrusions with a light directed from an incident light source having at least one excitation frequency;
wherein the biochemical molecules emit at least one surface enhanced Raman scattering (SERS) light in response to the light being directed onto the plurality of plasmonic metal protrusions from the incident light source;
detecting, by a detector, at least one laser power intensity and at least one Raman shift in vibrational wavenumber of the Raman dye of the at least one SERS spectra in a SERS spectra;
increasing a signal-to-noise ratio (SNR) of the at least one SERS spectra above a SNR predefined threshold by:
varying a thickness of a plasmonic metal in the plurality of plasmonic metal protrusions to be within a range between 20 nm to 200 nm;
varying a lateral size of each of the GO nanosheet coatings to be within a range between 43 nm and 295 nm; and
choosing a composition of the Raman dye to have a Raman cross-section value at the at least one excitation frequency greater than 3×10¹⁴ Hz;
receiving, by a processor, from the detector, data about the at least one Raman shift in vibrational wavenumber of the at least one SERS spectra, the at least one laser power intensity of the at least one SERS spectra, or any combination thereof; and
identifying, by the processor, the biochemical molecules in the sample based on the at least one Raman shift in vibrational wavenumber of the at least one SERS spectra, the at least one laser power intensity of the at least one SERS spectra, or any combination thereof.

17. The method according to claim 16, wherein the composition of the Raman dye comprises Cy5.

18. The method according to claim 16, wherein the biochemical molecules in the sample are selected from the group consisting of: cells, cell-derived vesicles, RNA sequences, DNA sequences, pathogens, antigens, viruses, and viral particles.

19. The method according to claim 16, wherein the substrate is glass.

20. The method according to claim 16, wherein the plasmonic metal is gold.

21. The method according to claim 20, further comprising coating the plurality of plasmonic metal protrusions with GO nanosheets by applying electrostatic interactions using a chemical linker; and wherein the plurality of plasmonic metal protrusions have a GO coating thickness in a range between 1 and 2 nm.

22. The method according to claim 20, further comprising forming the plurality of plasmonic metal protrusions on the substrate using laser interference lithography and a physical vapor deposition (PVD) of gold.

23. The method according to claim 22, wherein forming the plurality of plasmonic metal protrusions using the laser interference lithography and the PVD of gold comprises forming each of the plurality of plasmonic metal protrusions that are cone-shaped.

24. The method according to claim 23, wherein each cone-shaped plasmonic metal protrusion has a width of 250 nm and a height of 100 nm.

25. The method according to claim 16, wherein the biochemical molecules comprise neural stem cells, and further comprising monitoring, by the processor, changes in the SERS spectra for characterizing neural stem cell differentiation.
